(19) United States
(12) Patent Application Publication
(10) Pub. No.: US 2012/0070856 A1
(43) Pub. Date: Mar. 22, 2012

(54) CROSS-LINKING AGENTS

(75) Inventors: Jean Martinez, Caux (FR); Gilles Subra, Saint Gely Du Fesc (FR); Christine Goubet, Les Matelles (FR); David Paramelle, Saint Juery (FR); Eric Forest, Saint-Egreve (FR); Michaël Heymann, Illzach (FR); Christophe Geourjon, Lyon (FR)

(73) Assignees: Centre National De La Recherche Scientifique (CNRS); Universite Montpellier 2 Science ET Techniques; Universite De Montpellier I

(21) Appl. No.: 13/254,500

(22) PCT Filed: Mar. 4, 2010
(86) PCT No.: PCT/EP10/52773

§ 371 Date: Nov. 14, 2011

(30) Foreign Application Priority Data
Mar. 4, 2009 (EP) 09305201.7

(51) Int. Cl. C12Q 1/37; C07D 207/46; C07D 403/12

(52) U.S. Cl. 435/23; 548/520; 548/542

(57) ABSTRACT

The invention relates to a protein cross-linking agent of the formula, where R₁ is an aryl group optionally substituted once or several times by a grouping selected from the group consisting of hydroxy, C₁-C₄ alkyl, OBoc, SO₃Na, Deu, and C₁-C₄ alkoxy groupings, R₂ is N, (III), or (IV), n and in are identical or different integers between 0 and 10, p is an integer between 0 and 5, k is 0, 1, 2 or 3, and X and X′ are identical or different and are a reactive function of the proteins. The invention also relates to a method for the structural analysis of a protein or a protein complex.

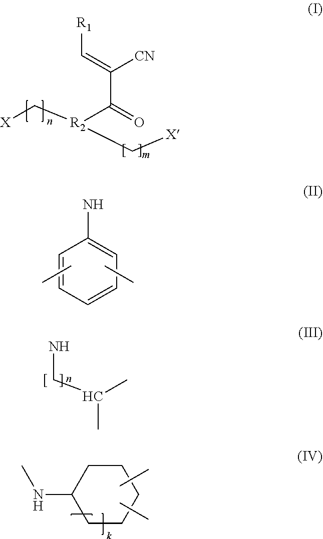

The invention relates to the field of structural analysis of proteins by mass spectrometry.

Analysis of the three-dimensional structures of proteins and their interactions with their environment (e.g., DNA, proteins, membranes) is very important for the understanding of the biological functions of proteins in their natural environment.

In order to obtain these structural data at high resolution, NMR and XRD are the analysis methods commonly used. However, these methods are highly limited by certain constraints, namely protein crystallization and the use of a large quantity of material (5 mg to 10 mg of proteins).

The use of chemical cross-linking agents together with mass spectrometry is an increasingly frequent method for determining low-resolution structural data free from existing constraints.

This approach involves carrying out an enzymatic digestion of the proteins from the reaction medium after having carried out the cross-linking reaction in solution. The peptide mixture obtained is then analyzed by mass spectrometry in order to identify the peptides modified by the cross-linking agents. Structural data are then obtained from this identification. This strategy has been used in the context of low-resolution structural studies of proteins or of protein-protein interactions.

Chemical cross-linking agents have one or two reactive functional groups connected by a spacer. These reactive functional groups are able to react with the side chains of protein amino acids. After identification of the protein amino acid residues modified by the two reactive functional groups, distance constraints within these proteins can be determined by the known length of the spacer of the chemical cross-linking agent.

Homobifunctional agents, such as bis-imidoesters and dialkyl halides, have been developed and used on proteins since the 1950s.

However, the use of protein cross-linking agents in solution poses a major problem in terms of the detection of peptides of interest in a complex mixed medium comprising both unmodified peptides and peptides modified by the cross-linking agents.

Indeed, to the significant number of types of cross-linking products is added the complexity of the peptide mixture resulting from enzymatic digestion. Consequently, the signal intensity of the peptides of interest is often very low. To overcome this difficulty, numerous strategies involving the labeling of chemical cross-linking agents have been developed. They allow either the enrichment of cross-linked peptides using purification methods or the discrimination of their signals during mass spectrometry analyses. These methods include:

Isotopic Labeling

The cross-linking experiment is carried out with an equimolar mixture of two cross-linking agents of identical structure but with one modified by stable isotopes. The isotopes used are deuterium and oxygen-18. This type of labeling will split the signal of the cross-linked peptide due to the difference in mass between the two types of cross-linking agents, thus facilitating the detection of modified peptides.

Cleavable Cross-Linking Agents

The use of a cleavable functional group within the cross-linking agent facilitates the identification of cross-linked peptides by comparison between the mass spectrum of all the signals of the peptides of interest and that obtained after cleavage of the cross-linking agent. It is thus possible to identify the signals that have been affected by cleavage and thus to deduce therefrom the masses of the products modified by the cross-linking agent.

Cleavage can be induced either by a chemical treatment or directly during analyses by tandem mass spectrometry (MS/MS).

Fluorescent Labeling

The purpose of using a cross-linking agent labeled with a fluorescent probe is to facilitate the detection of cross-linked peptides during chromatographic purification.

Affinity Purification

The labeling of a trifunctional cross-linking agent with biotin makes it possible to carry out affinity purification with avidin beads. This method thus makes it possible to enrich the sample in cross-linked peptides after the enzymatic digestion step.

UV-Absorbing Label

The use of peptide labeling with a UV-absorbing compound in order to improve detection in MALDI-TOF mass spectrometry was disclosed in application WO03/087839. This study showed that the labeling of peptides with HCCA can increase the intensity of their signals.

A need thus exists for a new method for improving the sensitivity of detection of peptides of interest in MALDI-TOF mass spectrometry and the discrimination of their signal in the case of complex mixtures, in particular for the low-resolution study of three-dimensional protein structures, for example by identifying distance constraints between various side chains.

The Applicant previously showed (Lascoux et al., Discrimination and Selective Enhancement of Signals in the MALDI Mass Spectrum of a Protein by Combining a Matrix-Based Label for Lysine Residues with a Neutral Matrix, Angew. Chem. 2007, 119, 5690-5693) that the joint use of α-cyano-4-hydroxycinnamic acid (HCCA) as peptide label and a neutral matrix of similar structure, α-cyano-4-hydroxycinnamate methyl ester (HCCE), during analyses by MALDI-TOF mass spectrometry, makes it possible to increase the signal of labeled peptides in a small proportion in a mixture of unlabeled peptides, and to obtain a signal discrimination effect called the “HCCA/HCCE discrimination effect.”

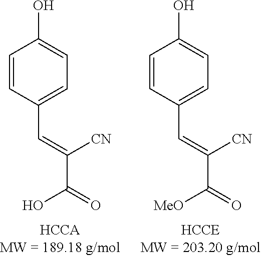

In an unexpected and surprising manner, the Applicant has now shown that bifunctional cross-linking agents labeled with HCCA or with an HCCA derivative make it possible to identify by MALDI-TOF mass spectrometry peptides of interest of a protein in a complex peptide mixture by labeling in solution, whereas said peptides are not visible on the MALDI-TOF mass spectrum of the protein. This selective effect is further accentuated by the use of HCCE matrix.

### SUMMARY OF THE INVENTION

The invention thus relates to a protein cross-linking agent of formula (I)

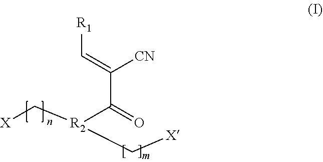

wherein
R₁ is an aryl group optionally substituted once or several times by a group selected from hydroxy, C₁-C₄ alkyl, OBoc, SO₃Na, Deu, C₁-C₄ alkoxy,

### R₂ is N,

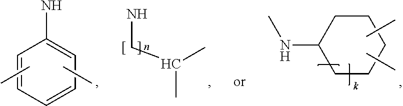

n and m are identical or different integers between 0 and 10, preferably between 1 and 5,
p is an integer between 0 and 5,
k is 0, 1, 2 or 3,
X and X′ are identical or different and are a protein reactive functional group.

The invention also relates to a method for preparing a cross-linking agent according to the invention.

The invention also relates to the use of a cross-linking agent according to the invention for the mass spectrometry analysis of the three-dimensional structure of a protein.

The invention also relates to a method for the structural analysis of a protein or a protein complex comprising the following steps:
a) cross-linking of the protein or protein complex on the cross-linking agent according to any one of claims 1 to 8 by the X and/or X′ functional groups,
b) enzymatic digestion of the protein or protein complex bound to the cross-linking agent according to any one of claims 1 to 8,
c) analysis by mass spectrometry.

### DETAILED DESCRIPTION

### Definitions

“CO₁₋₁₀ alkyl” group refers to a linear or branched saturated hydrocarbon chain comprising from 1 to 10 carbon atoms, such as, for example, a methyl, ethyl, isopropyl, tert-butyl or pentyl group, etc.

“C₂₋₁₀ alkenyl” group refers to a linear or branched hydrocarbon chain comprising at least one unsaturation and comprising from 2 to 10 carbon atoms, such as, for example, an ethenyl, propenyl or 2,4-hexadienyl group, etc.

“Aryl” group refers to an aromatic group, preferably comprising 5 to 10 carbon atoms, comprising one or more rings and optionally comprising a heteroatom, in particular oxygen, nitrogen or sulfur, such as, for example, a phenyl, furan, indole, pyridine or naphthalene group, etc.

The term “halogen” refers to fluorine, bromine, chlorine or iodine.

“Boc” refers to an amino-protecting group of formula t-butyloxycarbonyl.

“Deu” refers to deuterium.

In the context of the present invention, “(C₁-C₆)alkoxy” group refers to a (C₁-C₆)alkyl group, as defined above, bound to the molecule via an oxygen atom. Examples include methoxy, ethoxy, propoxy, isopropoxy, butoxy or tert-butoxy groups.

“tBu” refers to tert-butyl.

### Cross-Linking Agents

The protein cross-linking agent of the invention has formula (I)

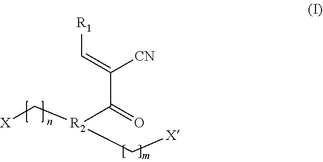

wherein
R₁ is an aryl group optionally substituted once or several times by a group selected from hydroxy, C₁-C₄ alkyl, OBoc, SO₃Na, Deu, C₁-C₄ alkoxy,

### R₂ is N,

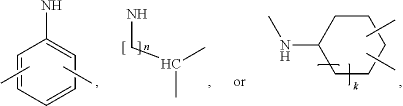

n and m are identical or different integers between 0 and 10, preferably between 1 and 5,
p is an integer between 0 and 5,
k is 0, 1, 2 or 3,
X and X′ are identical or different and are a protein reactive functional group.

Advantageously, n=m.

Preferably, R₁ is a phenyl group optionally substituted once or several times by a group selected from hydroxy, C₁-C₄ alkyl, OBoc, SO₃Na, Deu, C₁-C₄ alkoxy.

In a particularly preferred manner, R₁ is a phenoxy group, particularly a para-hydroxy-phenyl group.

Preferably, R₂ is N,

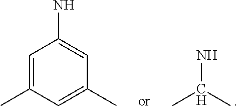

Advantageously, the cross-linking agent of the invention has formula (II), (III) or (IV)

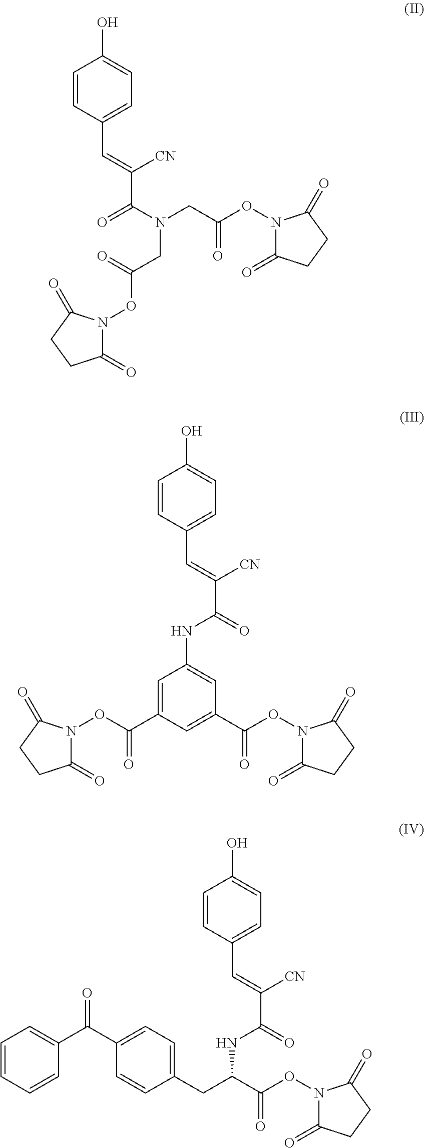

Advantageously, X and X′ are identical or different and are selected from the group comprised of imidoester, N-hydroxysuccinimide ester, isocyanate, isothiocyanate, N-maleimide, disulfide, 1,2-dicarbonyl, benzophenone and aryl azide functional groups.

The cross-linking agent of the invention comprises two protein reactive functional groups, X and X′. It is possible to conceive of a very wide variety of cross-linking agents according to the nature of the reactive functional groups used, which may or may not be identical (i.e., a homobifunctional or heterobifunctional agent).

“Cross-linking of the protein or protein complex on the cross-linking agent on the solid support of the invention” refers to the reaction of one or more X and/or X′ reactive functional groups of cross-linking agent A with one or more groups of the protein or protein complex resulting in the covalent bonding of said protein or said protein complex with cross-linking agent A.

Homobifunctional cross-linking agents have two identical reactive functional groups capable of reacting with the same type of functional group.

During protein cross-linking experiments, one of the two reactive functional groups reacts with a side chain of an amino acid residue. The second reactive functional group then reacts either intramolecularly on another neighboring side chain, or on a side chain of an amino acid residue belonging to another protein. Since the two reactive functional groups are identical, the reaction protocol involves a single step.

Heterobifunctional cross-linking agents have two reactive functional groups targeting different amino acids. They are generally used to cross-link proteins in two steps (e.g., cross-linking agents with a non-specific reactive functional group and a specific reactive functional group). Once the first bonding reaction is carried out, it is thus possible to purify the modified proteins before carrying out the reaction of the second reactive functional group. This can promote intramolecular reactions and can make it possible to obtain more diversified data than is possible with homobifunctional cross-linking agents.

### Reactive Functional Groups

X and X′ are specific protein reactive functional groups able to react with a protein reactive group.

Advantageously, X and X′ are identical or different and are selected from the group comprised of specific amine reactive functional groups, specific carboxylic acid reactive functional groups, specific thiol reactive functional groups, specific guanidine reactive functional groups, nonspecific reactive functional groups.

Specific amine reactive functional groups react with the primary amines present on proteins. There are two types of primary amines within a protein: N-terminal and lysine N-ε. Studies have shown that it is possible to target them independently due to their different pKa values (pKa_{N-terminal}=8 and pKa_{lysine N-ε}=10.5).

Three types of functional groups are commonly used to this end: imidoesters, N-hydroxysuccinimide (NHS) esters and isocyanates (and isothiocyanates). All these are activated electrophiles possessing a good leaving group by nucleophilic substitution.

Specific carboxylic acid reactive functional groups are present in the residues Asp and Glu and partly in the C-terminal of proteins. These functional groups are not reactive themselves and require activation. This activation is generally carried out with carbodiimides. The O-acylurea formed by this activation makes it possible to react with a primary amine and to form an amide bond.

Specific thiol reactive functional groups are the N-maleimide and disulfide functional groups. The thiol functional group is the most reactive nucleophilic functional group within a protein. However, the side chains of cysteine residues are often engaged in disulfide bridges, thus preventing their reaction with chemical cross-linking agents. Consequently, it is generally necessary for these bonds to undergo a reduction reaction (with ethanedithiol, or EDT) in order to find free thiol functional groups. Since the pKa of cysteine residue thiols is approximately 8.6, their reactivity increases when the thiolate ion is formed at pH greater than 8.6.

Specific guanidine reactive functional groups are the 1,2-dicarbonyl compounds which react specifically with arginine side chains.

Nonspecific reactive functional groups react with molecules by exposure to UV light. An ideal photoreactive agent must have various qualities:
It has strong reactivity;
It is able to react indifferently on any type of residue;
It is stable in the dark;
It reacts with light whose wavelength does no photolytic damage to the biological sample;
Its reaction product must be stable.

The reaction mechanisms of these functional groups generally involve radicals, thus making it possible to act indifferently on various residues. Generally, chemical cross-linking agents with a non-specific reactive functional group also have a specific reactive functional group. In this manner, it is possible to target residues of interest with a first bonding step involving the specific reactive functional group and then to mark all the residues present within a certain perimeter (defined by the length of the spacer linking the two reactive functional groups) via the action of the non-specific reactive functional group.

There are two types of functional groups commonly used to this end: benzophenones and aryl azides.

### Method of Preparation

The cross-linking agents of the invention are preferentially prepared in the following way.

The method for preparing the cross-linking agents of the invention comprises three steps.

The first step of these three steps consists of step (a) or (a′).

Step (a) consists of peptide coupling between the amine RR′NH and the carboxylic acid

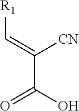

whose acid functional group is optionally activated, to give

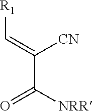

with R₁ as previously defined,
with R and R′:

each representing -(alkyl)-COOY, or

one representing H and the other

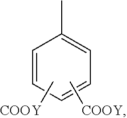

or

one representing H and the other

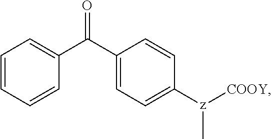

with Y representing H or tBu,
and with Z representing a C₁-C₈ alkyl group.

Step (a′) comprises steps (i), (ii) and (iii).

Step (i) consists of the reaction of RR′NH with

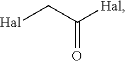

where Hal is a halogen atom,
in the presence of a base to give

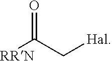

Step (ii) consists of the reaction of

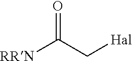

with a cyanide, such as KCN, to give

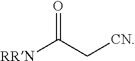

Step (iii) consists of the reaction of

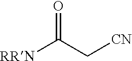

with R₁CHO in the presence of a base to give

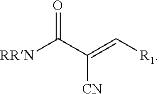

Step (b) consists of the optional hydrolysis of the ester obtained in step (a) or (a′) in acid to give

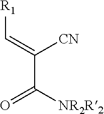

with R₂ and R₂′:

each representing -(alkyl)-COOH, or

one representing H and the other

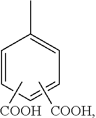

or

one representing H and the other

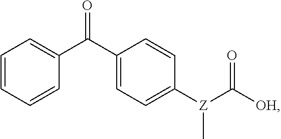

with Z representing a C₁-C₈ alkyl group.

Step (c) consists of the reaction of the compound obtained in the preceding step (b) to obtain a protein reactive functional group.

This protein reactive functional group is preferentially an NHS ester functional group.

Peptide coupling with carboxylic acid is carried out preferably in the presence of a coupling agent, such as diisopropylcarbodiimide (DIC), dicyclohexylcarbodiimide (DCC), 1-(3-dimethylaminopropyl)-3-ethylcarbodiimide hydrochloride (EDC), carbonyldiimidazole (CDI), 2-H-benzotriazole-1-yl)-1,1,3,3-tetramethyluronium hexafluorophosphate (HBTU), 2-(1H-benzotriazole-1-yl)-1,1,3,3-tetramethyluronium tetrafluoroborate (TBTU) or O-(7-azobenzotriazol-1-yl)-1,1,3,3-tetramethyluronium hexafluorophosphate (HATU), optionally combined with a coupling aid such as N-hydroxysuccinimide (NHS), N-hydroxybenzotriazole (HOBt), 3,4-dihydro-3-hydroxy-4-oxo-1,2,3-benzotriazole (HOOBt), 1-hydroxy-7-azabenzotriazole (HOAt) or N-hydroxysulfosuccinimide (sulfo-NHS).

Amine functional group protection/deprotection reactions and peptide coupling reactions are well known to those persons skilled in the art.

### Method of Protein Cross-Linking in Solution for Structural Analysis by Mass Spectrometry

Protein Cross-Linking

The chemical cross-linking agent in solution is brought together with the dissolved protein.

Advantageously, the supported chemical cross-linking agent is brought together with the dissolved protein in a saline buffer in order to approximate physiological conditions.

The result is covalent bonding of the protein with the cross-linking agent via the X and/or X′ reactive functional groups.

Enzymatic Digestion

The cross-linked proteins are digested as follows: the mixture is brought together with a solubilized enzyme (e.g., trypsin), preferably in a saline buffer.

Analysis by Mass Spectrometry

### Mass Spectrometry

Preferably, the mass spectrometry analysis is carried out by MALDI-TOF spectrometry.

The samples are deposited using either the dried droplet method or the thin layer method.

The dried droplet method is preferred and is carried out by co-crystallization of a droplet of matrix solution and a droplet of analyte solution.

### Mass Spectrometry Data Analysis Software

Advantageously, the data from the mass spectrometry analysis of the peptide mixtures are analyzed using software.

Indeed, mass spectrometry analysis of the peptide mixtures provided by the enzymatic digestion step generally generates a great quantity of data. In order to facilitate the identification of peptides detected during such experiments, numerous prediction software packages have been created (e.g., Pro-CrossLink, VirtualMSLab, SearchXLink and MS2Assign).

The goal of these software packages is to quickly provide a list of potential peptides that are not modified or that are modified by the cross-linking agent used, corresponding to each m/z value of the mass spectrum. The experimental values are compared with the theoretical values calculated from an in silico digestion of the protein model studied.

In the context of the present invention, a prediction software package, called MSX-3D, was created at the Institute of Biology and Chemistry of Proteins (IBCP) in Lyon, France.

### MALDI-TOF Matrices

HCCA matrix is commonly used during MALDI-TOF mass spectrometry analyses of peptides. It is able to absorb UV light from the laser of the MALDI source. The energy that it absorbs is released in the form of thermal energy and enables desorption of compounds co-crystallized with the matrix.

α-Cyano-4-hydroxycinnamate methyl ester, called HCCE or α-CNME, is a matrix not often used in MALDI-TOF mass spectrometry analysis. It is the methyl ester of HCCA matrix. Due to their closely related structures, they share similar characteristics:
Molecular weights (189 g/mol for HCCA and 203 g/mol for HCCE);
Same physical appearance: yellow crystalline powder;
Similar aromatic structures give them comparable absorption properties.

However, there is an important factor that differentiates them: their ability to give a proton. Indeed, HCCA matrix has two acid functional groups of distinct pKa, the carboxylic acid functional group (pKa₁=2) and the phenol alcohol functional group (pKa₂=8). On the other hand, the HCCE matrix has only one acid functional group, the phenol alcohol functional group (pKa₃=8).

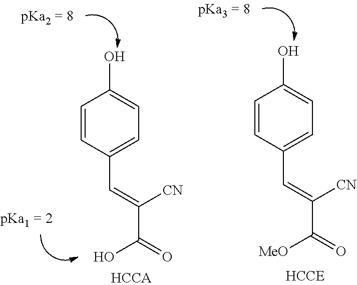

HCCA matrix thus has a carboxylic acid functional group allowing it to play the part of proton donor during the ionization process of the MALDI source, which is why it is called an acidic matrix. HCCE matrix is much less effective in this role because it has only one weakly acidic functional group (i.e., phenol alcohol, pKa₃=8) and is thus called a neutral matrix.

### FIGURES

FIG. 1: Analysis of an equimolar mixture of five model peptides JMV3346 to JMV3350 with HCCA matrix.

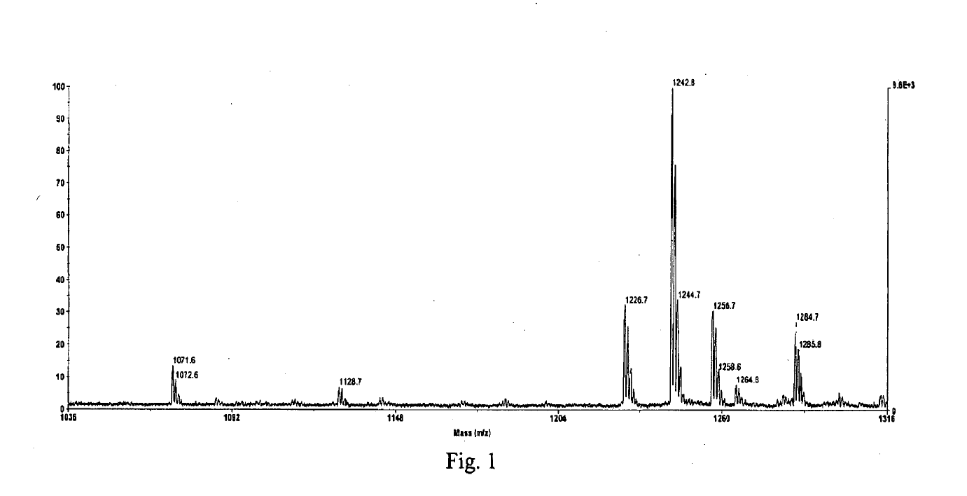

FIG. 2: Mass spectrum of the peptide mixture resulting from trypsin digestion of unmodified cytochrome c (with HCCA matrix).

FIG. 3: Mass spectrum of the trypsin digestion after cross-linking of cytochrome c (100 μM) with R=0.5 (with HCCA matrix): [symbol] peptide signal potentially modified by JMV3378.

FIG. 4: Mass spectrum of the trypsin digestion after cross-linking of cytochrome c (100 μM) with R=0.5 (with neutral HCCE matrix): [symbol] peptide signal potentially modified by JMV3378.

FIG. 5: Mass spectrum of the peptide mixture resulting from trypsin digestion of unmodified apomyoglobin (with HCCA matrix).

FIG. 6: Mass spectrum of the trypsin digestion after cross-linking of apomyoglobin (100 μM) with R=0.2 (with HCCA matrix): [symbol] peptide signal potentially modified by cross-linking agent JMV3378.

FIG. 7: Mass spectrum of the trypsin digestion after cross-linking of apomyoglobin (100 μM) with R=0.2 (with neutral HCCE matrix): [symbol] peptide signal potentially modified by cross-linking agent JMV3378.

The following examples illustrate the invention without limiting its scope.

### EXAMPLES

### Example 1

### Influence of the Alcohol Functional Group of the HCCA Label

The five model peptides are decapeptides modified by C-terminal amidation and possess the same amino acid sequence with the aim of not influencing their intrinsic ionization ability in MALDI-TOF mass spectrometry. They were synthesized on a solid support using the Fmoc strategy. The first peptide, JMV3346, is not labeled and is used as the reference to evaluate the effect of spectral discrimination. Model peptides JMV3347, JMV3348 and JMV3349 were synthesized by coupling HCCA and two derivatives in position 4 (i.e., α-cyanocinnamic acid, or CCA, and α-cyano-4-methoxycinnamic acid) with the decapeptide entirely protected and bound to the solid support. Peptide JMV3350 was obtained by acetylation of the phenol alcohol functional group of HCCA coupled with the decapeptide entirely protected and bound to the solid support.

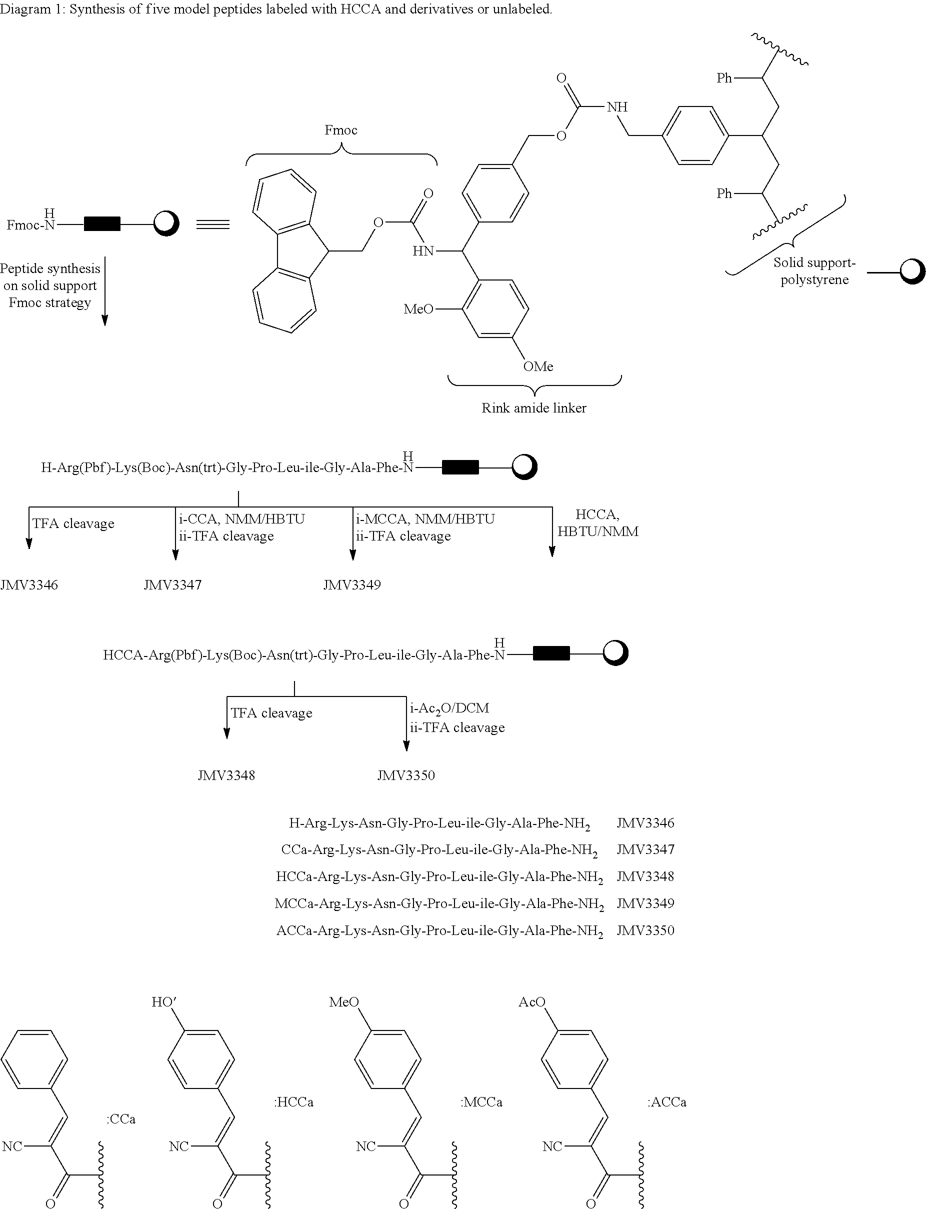

To that end, an equimolar mixture of the five model peptides (JMV3346 to JMV3350) was analyzed with HCCA matrix.

This analysis shows that the HCCA label provides an optimal increase in signal intensity. Moreover, despite a slight improvement in detecting peptides JMV3347, JMV3349 and JMV3350 in relation to the unlabeled peptide, it is observed that the absence of the phenol alcohol functional group or its modification by methylation or acetylation causes a strong reduction (approximately ⅔ of the signal in relation to the JMV3348 signal) in the detection of labeled peptides. This reduction can be due to a higher hydrophobicity of CCA, MCCA and ACCA labels induced by the loss of the proton of the alcohol functional group.

### Example 2

### Development of HCCA-Labeled Bifunctional Cross-Linking Agents

### 2-1—Synthesis of a Homobifunctional Agent

2-1-a—Agent JMV3378 with an Iminodiacetic Moiety

N-hydroxysuccinimidyl N-(α-cyano-4-hydroxycinnamyl)iminodiacetate (JMV3378) was prepared (Formula (II)).

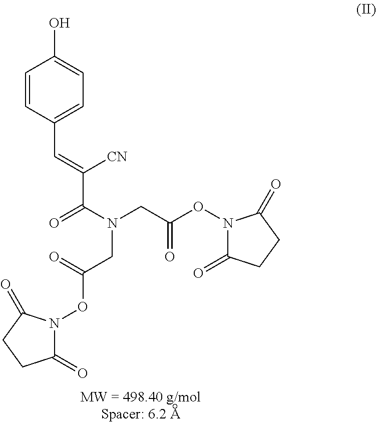

The direct coupling between the carboxylic acid functional group of HCCA and the secondary amine of diethyl iminodiacetate ester was first envisaged in order to synthesize JMV3378.

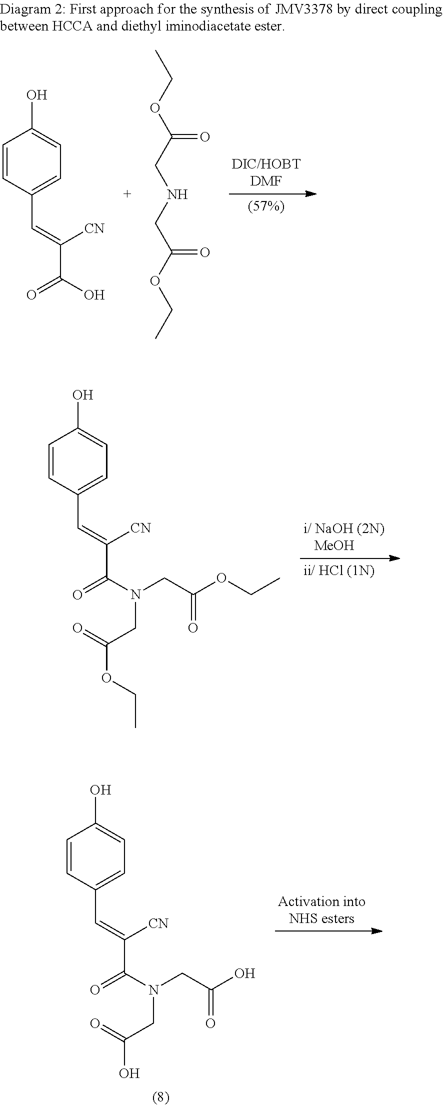

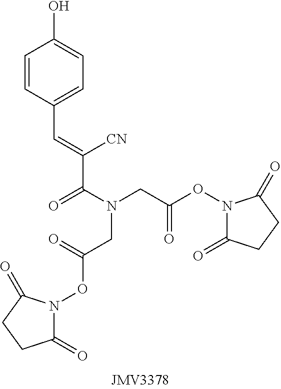

This synthesis route failed. Indeed, the saponification step led to degradation of the starting diester.

In order to avoid the ethyl ester saponification step, a new approach using tert-butyl esters was envisaged (see diagram 3). These esters are easily hydrolysable in trifluoroacetic acid (TFA) solution. Contrary to basic treatments, HCCA does not appear to be sensitive to TFA. Indeed, preparations of samples with HCCA in MALDI-TOF mass spectrometry as well as the HCCA-labeled model peptides (JMV3348) supported the use of concentrated TFA solutions.

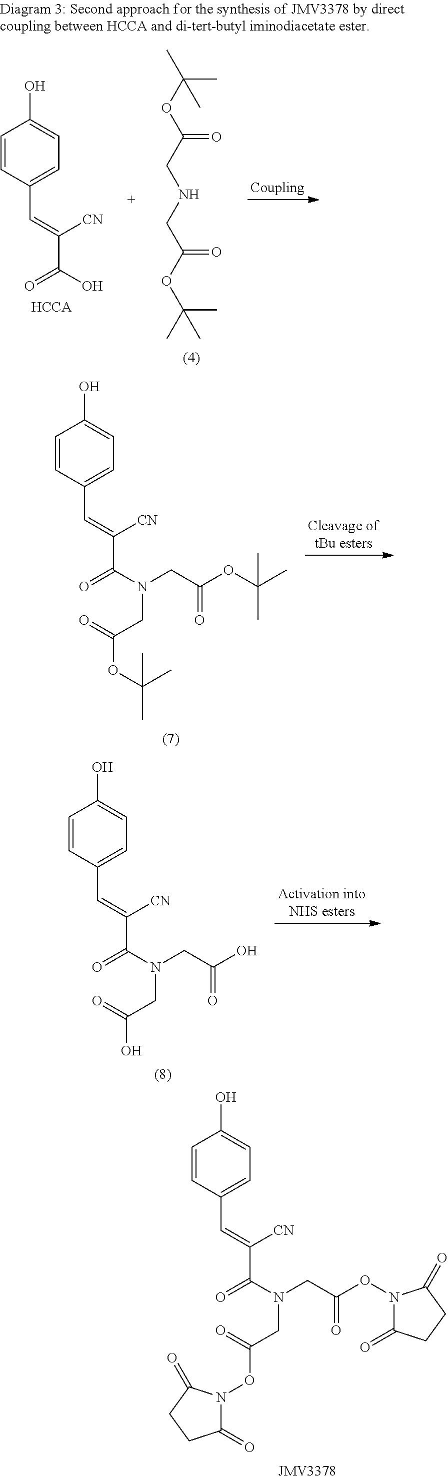

The key compound of this approach is di-tert-butyl iminodiacetate ester 4. Two synthesis routes were developed (see diagram 4).

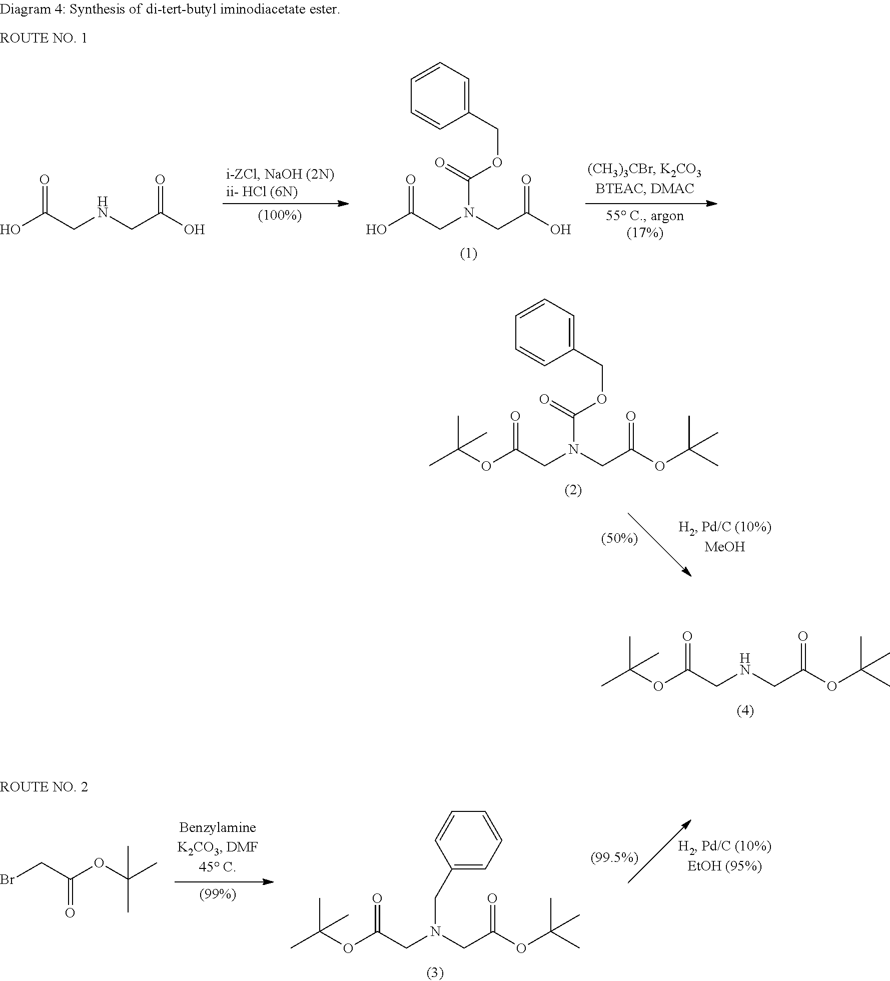

Route no. 1 comprises three steps. The secondary amine of iminodiacetic diacid is first protected with a benzyloxycarbonyl group in order to obtain compound 1. This reaction is carried out using a solution of benzyl chloroformate in soda with a quantitative yield. An esterification reaction of compound 1 carried out at 55° C. in dimethylacetamide (DMAC) solution containing benzyltriethylammonium chloride, potassium bicarbonate and tert-butyl bromide made it possible to obtain compound 2 with a yield of 17%. Finally, deprotection of the amine leading to compound 4 was carried out with a yield of 50% by hydrogenolysis in methanol with bubbling of hydrogen catalyzed by palladium on carbon.

Route no. 2 comprises only two steps. Compound 3 is obtained directly by double nucleophilic substitution of the primary amine of benzylamine on two molecules of tert-butyl 2-bromoacetate. This reaction is carried out with a yield of 99% in dimethylformamide (DMF) solution containing potassium bicarbonate at 45° C. Hydrogenolysis of the benzyl group leading to compound 4 was carried out quantitatively using bubbling of hydrogen and palladium on carbon in 95% ethanol.

Route no. 2 appears to be more advantageous because it produces the final diester 4 in two steps with very good yield and uses a commercial tert-butyl ester. Preparation of tert-butyl ester is the limiting step in route no. 1. Moreover, this route has an additional step and does not produce the diester with as good a yield as does route no. 2.

The development of these two routes made it possible to consider a synthesis route for obtaining homologous agents (see diagram 5). In this case, the esterification step is necessary because higher homologues of 2-bromoacetate tert-butyl esters are not commercially available.

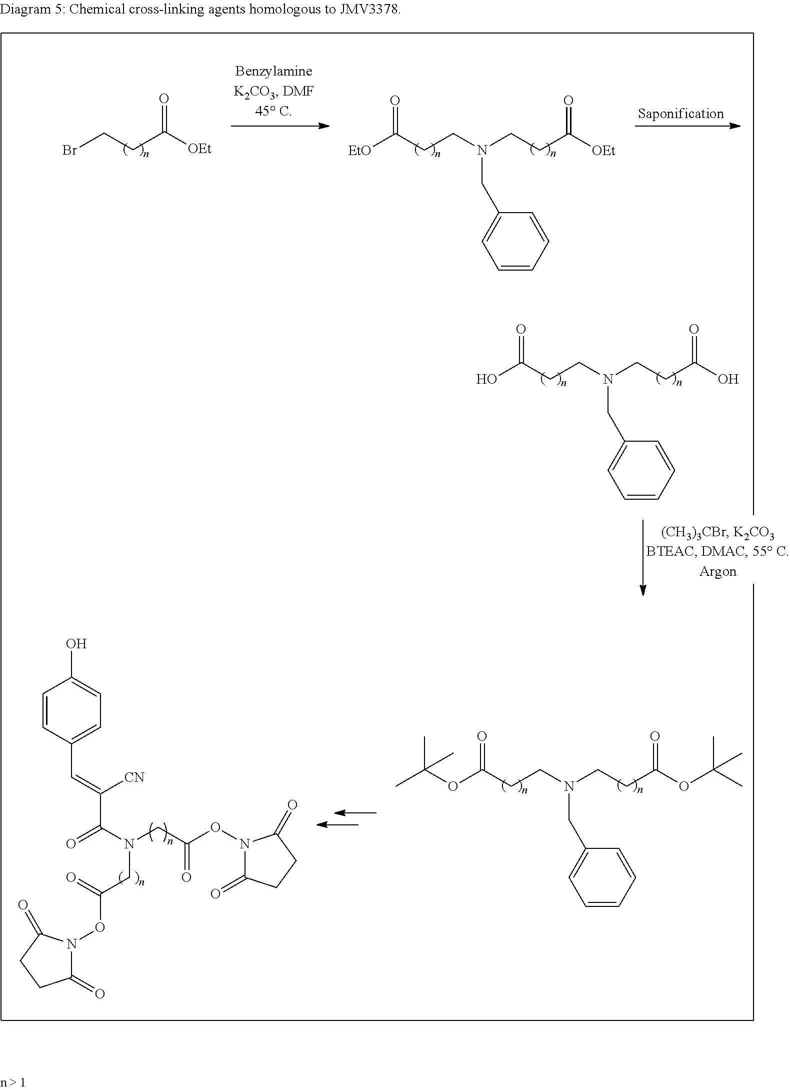

The synthesis of compound 7 was first envisaged by direct coupling between HCCA and the secondary amine of compound 4 (see diagram 6).

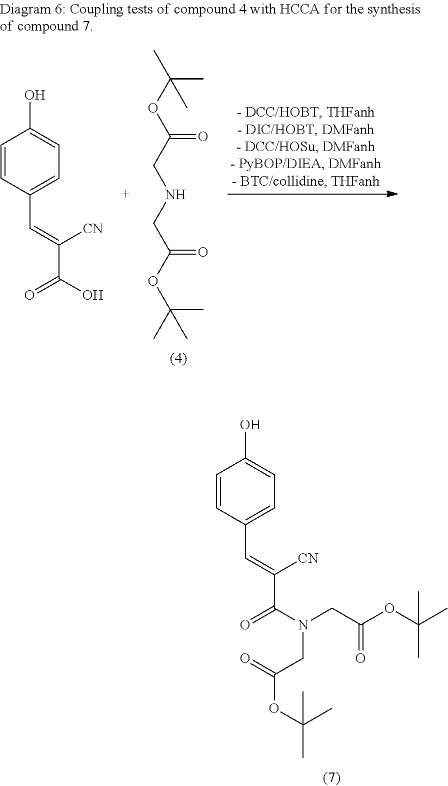

Various coupling methods were tested but none led to the formation of compound 7. Two hypotheses can be set forth to explain this failure. Obstruction of the two tert-butyl esters can make amine coupling difficult. Additionally, the phenol alcohol of HCCA can potentially be engaged in secondary intermolecular reactions and react with an active ester of another molecule.

Another approach was thus developed based on Knoevenagel condensation. This time, the HCCA moiety was synthesized in three steps from compound 4 (see diagram 7).

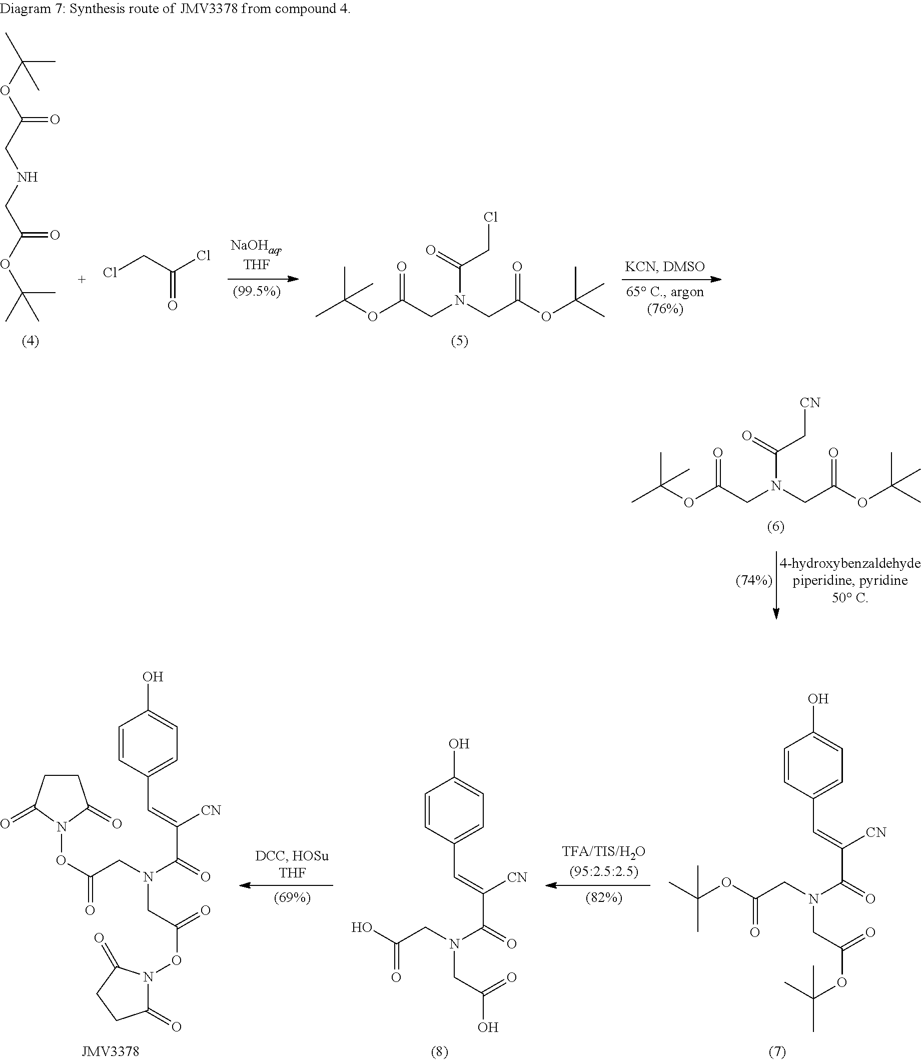

Compound 4 is first acylated with 2-chloroacetyl chloride in a mixture of soda and tetrahydrofuran (THF) in a quantitative manner. The substitution of chlorine with potassium cyanide in dimethyl sulfoxide (DMSO) at 65° C. results in compound 6. A Knoevenagel reaction between compound 6 and 4-hydroxybenzaldehyde carried out in a mixture of pyridine and piperidine at 50° C. provides compound 7 with a yield of 74%. During this reaction, aldehyde can be used in large excess in order to optimize the yield of the reaction. Excess aldehyde can be easily removed at the end of the reaction by liquid/liquid extraction in an aqueous phase saturated with sodium sulfite acidified by the addition of acetic acid (pH ˜5). The tert-butyl esters of compound 7 are hydrolyzed in a TFA/TIS/water mixture (95:2.5:2.5) to obtain diacid 8 with a yield of 82%. Triisopropylsilane (TIS) is a scavenger of tert-butyl carbocations. It is used to avoid a secondary Friedel-Crafts alkylation reaction at position 3,3′ of the phenol. Finally, JMV3378 is synthesized by activation of the two carboxylic acids with dicyclohexylcarbodiimide (DCC) and N-hydroxysuccinimide in THF with a yield of 69%. This compound is purified under the same conditions as those of JMV3155.

Chemical cross-linking agent JMV3378 was thus obtained via a seven-step synthesis route with a total yield of 31%.

2-1-b—Chemical Cross-Linking Agent with a 5-Aminoisophthalic Moiety

A second homobifunctional cross-linking agent with two NHS ester functional groups was synthesized: N-hydroxysuccinimidyl 5-(α-cyano-4-hydroxycinnamido)isophthalate (see diagram 8). This cross-linking agent has a spacer of similar length to that of compound JMV3378, however its structure is rigid because of the isophthalic aromatic ring. This rigidity makes it possible to obtain new structural information during protein cross-linking experiments.

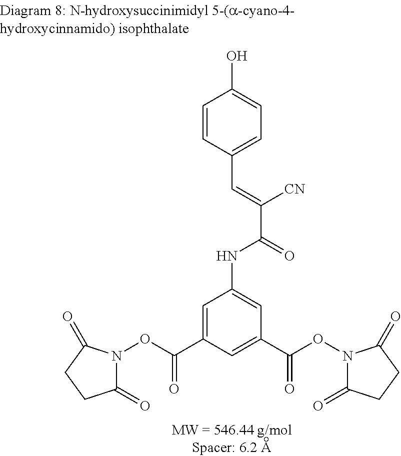

In order to synthesize this compound, a route similar to that developed for the synthesis of JMV3378 was used (see diagram 9).

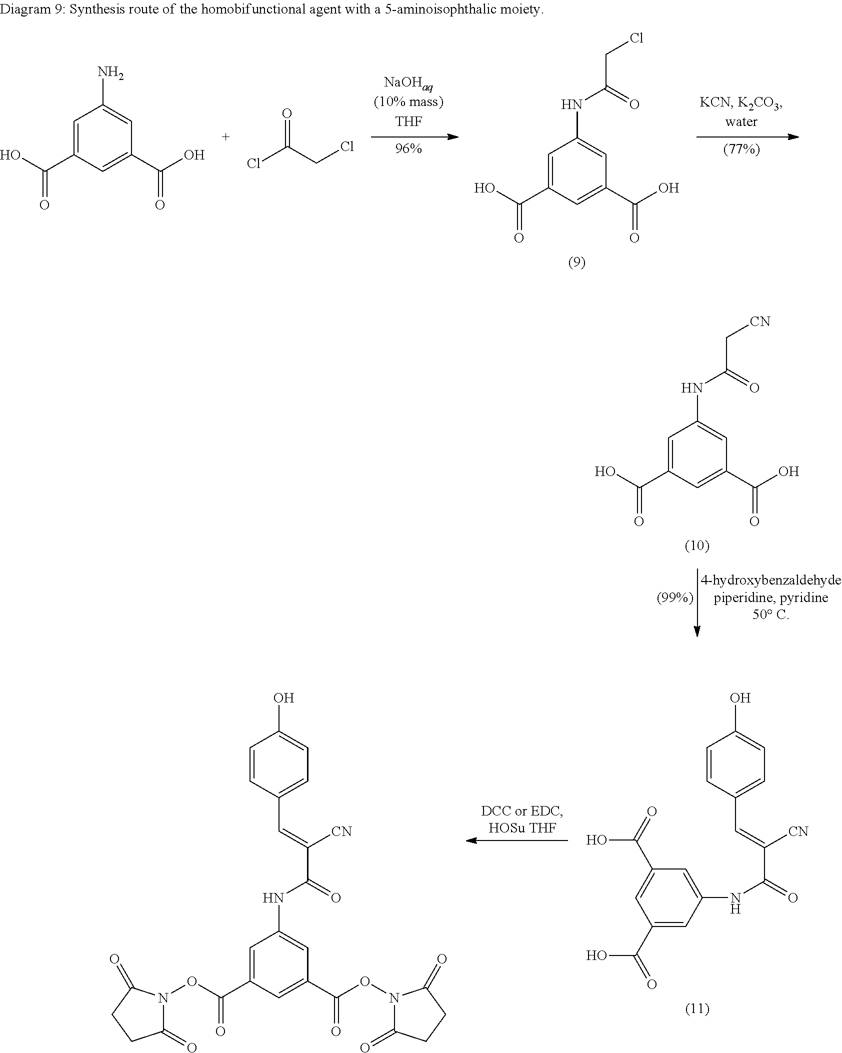

The synthesis route is initiated directly by acylation of 5-aminoisophthalic diacid with 2-chloroacetyl chloride in a mixture of soda and THF. Compound 9 is recovered by instantaneous precipitation in petroleum ether with a yield of 96%. Substitution of the chlorine atom of compound 9 with potassium cyanide in water in the presence of potassium bicarbonate results in compound 10 with a yield of 77%. Diacid 11 is then obtained with a yield of 99% via a Knoevenagel reaction with 4-hydroxybenzaldehyde in a mixture of pyridine and piperidine at 50° C.

Finally, the compound is synthesized by activation of the two carboxylic acids with dicyclohexylcarbodiimide (DCC) and N-hydroxysuccinimide in THF. This compound is purified under the same conditions as those of JMV3155.

### 2-2—Synthesis of a Heterobifunctional Agent

A heterobifunctional cross-linking agent with a nonspecific reactive functional group makes it possible to obtain much more diversified information than a homobifunctional cross-linking agent.

A cross-linking agent with both an NHS ester functional group and a photoactivatable benzophenone functional group was synthesized: N-hydroxysuccinimidyl (S)-3-(4-benzoylphenyl)-2-(α-cyano-4-hydroxycinnamido)propanoate, or JMV3480 (see diagram 10).

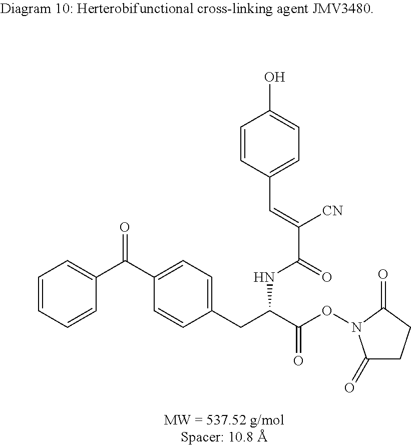

The synthesis route of this compound is presented in diagram 11 below.

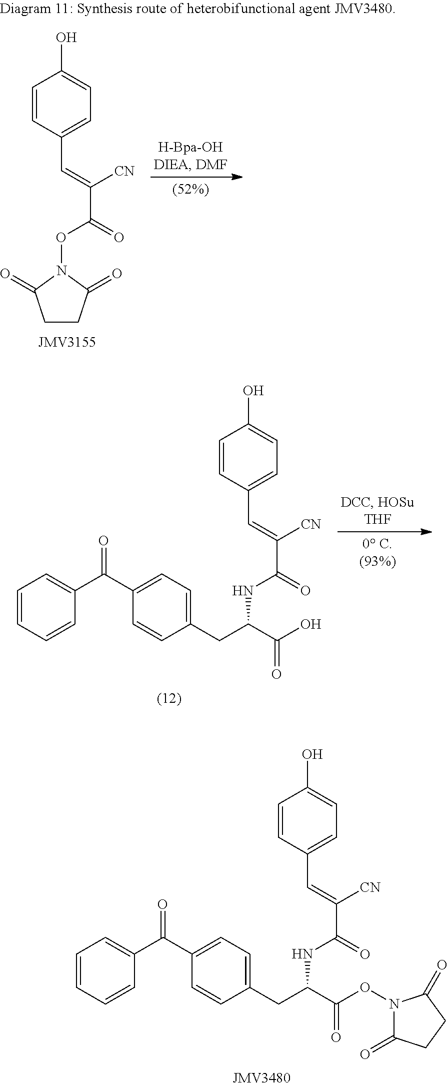

Amino acid coupling between monofunctional agent JMV3155 and (S)-p-benzoylphenylalanine acid in DMF solution containing N,N-diisopropylethylamine (DIEA) led to compound 12 with a yield of 52%. Finally, activation of the acid functional group into NHS ester leading to JMV3480 was carried out with DCC and N-hydroxysuccinimide in THF at 0° C. with a yield of 93%.

These various syntheses thus made it possible to obtain two new chemical cross-linking agents labeled with the UV-absorbent HCCA moiety: a first homobifunctional agent with two NHS ester functional groups specific to primary amines, JMV3378, and a heterobifunctional agent with a NHS ester functional group and a nonspecific benzophenone functional group, JMV3480.

### Example 3

### Applications of Cross-Linking Agent JMV3378 to the Study of Model Proteins

Cytochrome c and apomyoglobin from equine heart were used as model proteins.

Several cross-linking experiments, varying protein concentration and molar ratio R=[JMV3378]/[Lys+Nter], were carried out in order to evaluate the optimal conditions for obtaining both a satisfactory degree of cross-linking and a good cross-linked peptide detection discrimination effect in relation to unmodified peptides after enzymatic digestion.

For each test, the protein was dissolved in pH 7.5 PBS buffer and the cross-linking agent was dissolved in DMSO. Compound JMV3378 was introduced in the protein medium with a 95:5 PBS/DMSO volume ratio in order to not disturb the structure of the protein. Each cross-linking experiment was carried out at room temperature for 2 minutes and the product was then frozen or directly analyzed.

After cross-linking, the protein samples were analyzed by MALDI-TOF mass spectrometry using sinapinic acid matrix prepared by the dried droplet method. After verification of the degree of cross-linking obtained, the samples were digested with trypsin. Each peptide mixture obtained was analyzed by mass spectrometry with HCCA matrix prepared by the dried droplet method and with HCCE matrix prepared by the thin layer method.

3-1—Application to the Study of Cytochrome c

Evaluation of the HCCA/HCCE Discrimination Effect

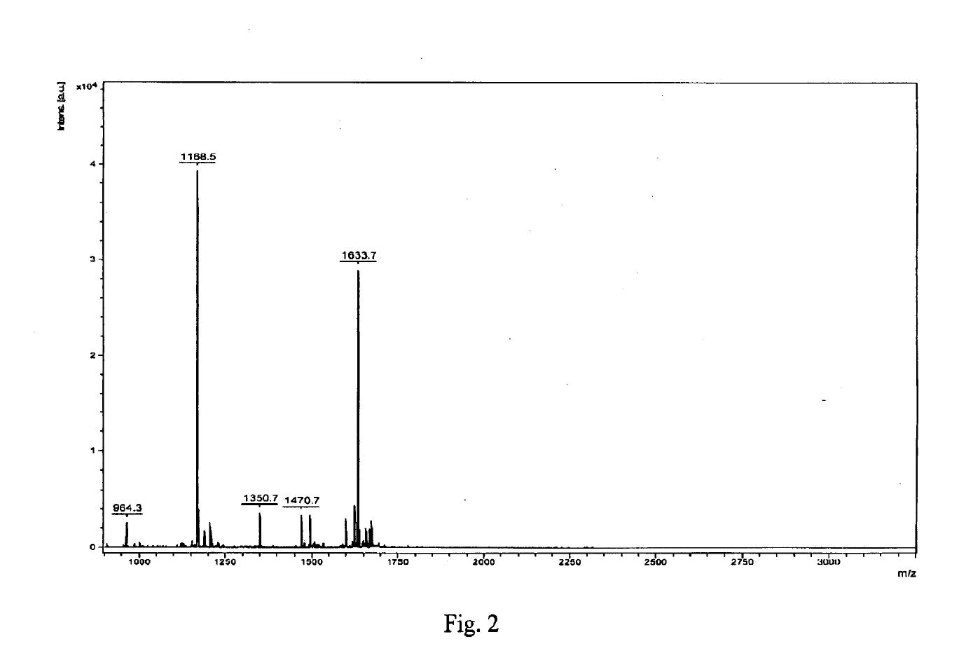

A reference MALDI-TOF mass spectrum of unmodified cytochrome c digested with trypsin and carried out with HCCA matrix prepared by the dried droplet method is presented below (see FIG. 2).

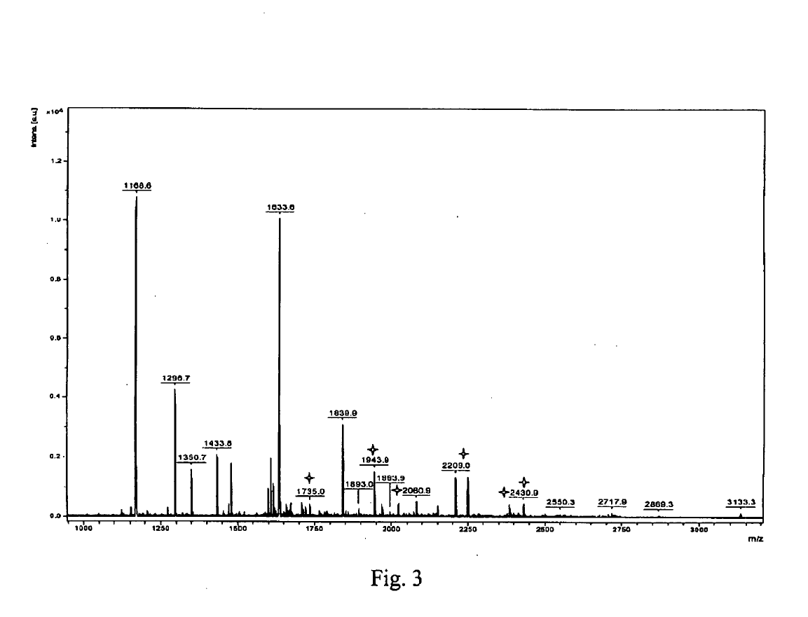

The mass spectrum of the peptide mixture resulting from trypsin digestion of the preceding cross-linked sample ([Cytoc]=100 μM and R=0.5) prepared in HCCA matrix by the dried droplet method is presented in FIG. 3.

Six signals not apparent on the unmodified cytochrome c digestion reference spectrum are visible on this spectrum with a rather low relative intensity. Signals corresponding to unmodified peptides (e.g., m/z: 1296 Da, 1633 Da, 1840 Da) are detected with relative intensities higher than those of the newly-appeared signals.

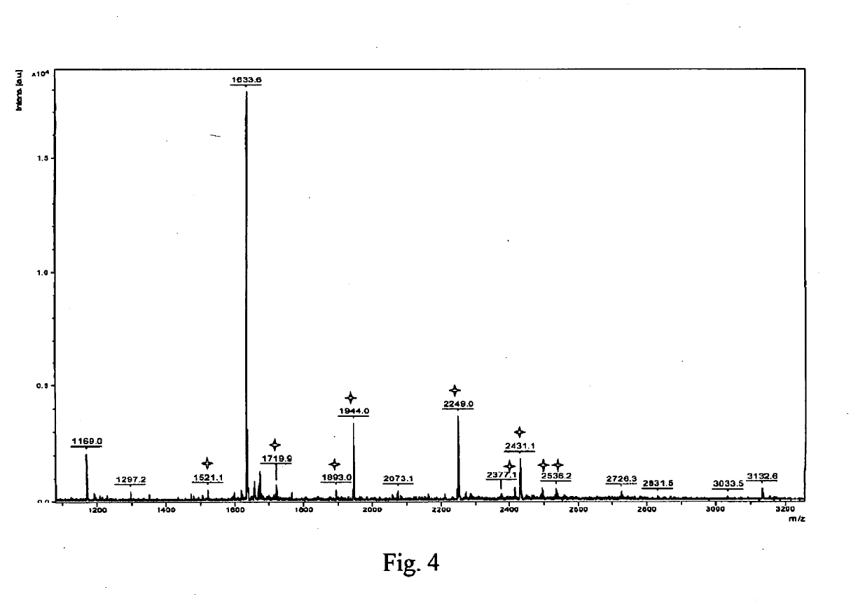

The same sample was analyzed using neutral HCCE matrix prepared by the thin layer method. The spectrum obtained is presented in FIG. 4 below.

This time the spectrum shows the appearance of nine signals not detected in the unmodified cytochrome c digestion reference spectrum, thus three more than with HCCA matrix. Moreover, certain ions are detected with a better resolution than when HCCA matrix is used (e.g., m/z: 1944 Da and 2249 Da). Finally, many ions corresponding to unmodified peptides are poorly visible when HCCE matrix is used (e.g., m/z: 1297 Da, 1351 Da, 1433 Da, 1840 Da). Only the m/z=1633 Da ion corresponding to unmodified peptide pre-charged by heme complexing an iron atom is still detected with good resolution.

This analysis thus shows that cross-linking with JMV3378 results in a satisfactory HCCA/HCCE discrimination effect and facilitates the detection of modified peptides of interest.

The list of these new ions detected was analyzed by MSX-3D prediction software. The results are presented in the following table.

TABLE 1
List of experimental and predicted masses
of peptides cross-linked by JMV3378.

| | | Devia- | | | |
|---|---|---|---|---|---|
| [M + H]⁺ | | tion | Sequence | Sequence | Modifi- |
| Experimental | Calculated | (ppm) | 1 | 2 | cations |
| 1521.06 | 1519.72 | −879 | 6-13 | | 2 × T0 |
| 1719.87 | 1719.84 | −20 | 26-38 | | T0 |
| 1893.01 | 1892.98 | −18 | 87-99 | | T0 |
| | 1892.98 | −18 | 88-91 | 92-100 | T2 |
| | 1892.98 | −18 | 88-100 | | T0 |
| 1943.99 | 1943.96 | −14 | 23-38 | | T1 |
| 2249.01 | 2248.03 | −435 | 23-38 | | 2 × T0 |
| | 2248.99 | −7 | 9-22 | | — |
| | 2249.11 | 45 | 73-87 | | T0 + T1 |
| | 2249.11 | 45 | 74-88 | | T0 + T1 |
| 2413.07 | 2413.04 | −14 | 39-55 | | 2 × T0 |
| 2495.29 | 2495.18 | −44 | 56-73 | | T0 |
| | 2495.35 | 23 | 73-79 | 80-91 | T2 |
| | 2495.35 | 23 | 73-86 | 87-91 | T2 |
| | 2495.35 | 23 | 73-87 | 88-91 | T2 |
| 2536.18 | 2535.05 | −444 | 9-22 | | T0 |
| | 2536.34 | 64 | 6-13 | 80-88 | T0 + T2 |

The detected signals can correspond to various types of peptide cross-links (types 0, 1 and 2). After having verified the theoretical distances between the lysine side chains of cytochrome c, a total of eight possibilities of types 1 and 2 cross-linking reactions were noted (see diagram 12).

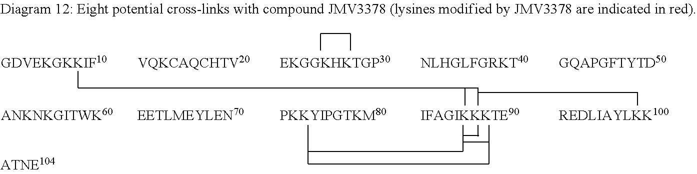

These results demonstrated that certain lysine side chains (Lys 5, 55, 100) not having reacted with compound JMV3378 are relatively inaccessible.

3-2—Application to the Study of Apomyoglobin

Cross-linking experiments with compound JMV3378 were carried out with apomyoglobin. The tests and the MALDI-TOF mass spectrometry analyses were carried out according to same protocols as for the experiments carried out on cytochrome c.

Evaluation of the HCCA/HCCE Discrimination Effect

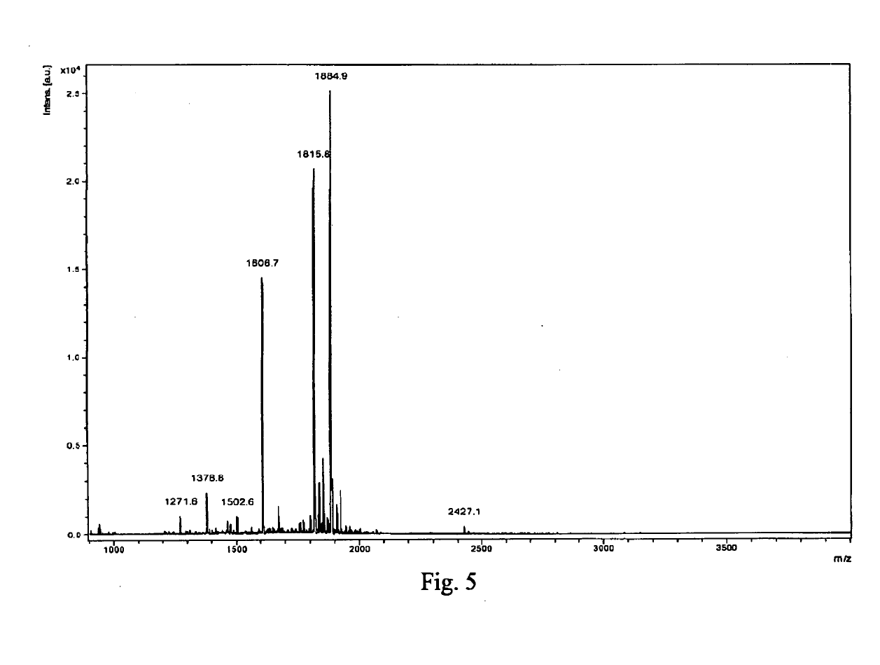

In point of comparison for the spectra obtained with the cross-linking experiment enzymatic digestions, the analysis of a peptide mixture resulting from trypsin digestion of unmodified apomyoglobin is presented in FIG. 5 below.

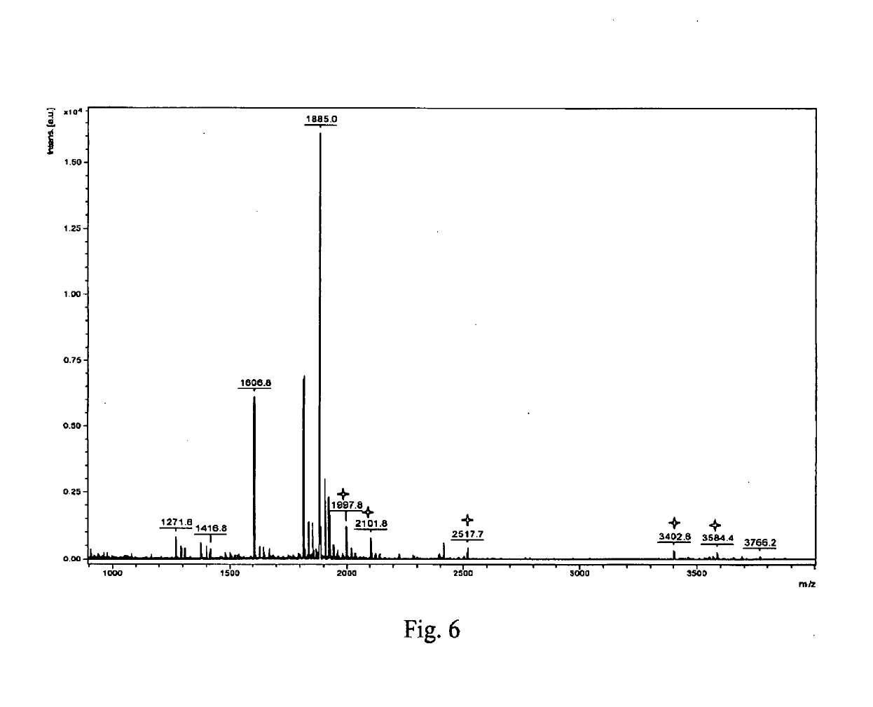

The preceding cross-linking experiment was digested with trypsin and then analyzed first with acidic HCCA matrix (see FIG. 6).

Direct comparison with the analysis of the peptide mixture resulting from digestion of native apomyoglobin shows that five new relatively weak signals are observed with HCCA matrix.

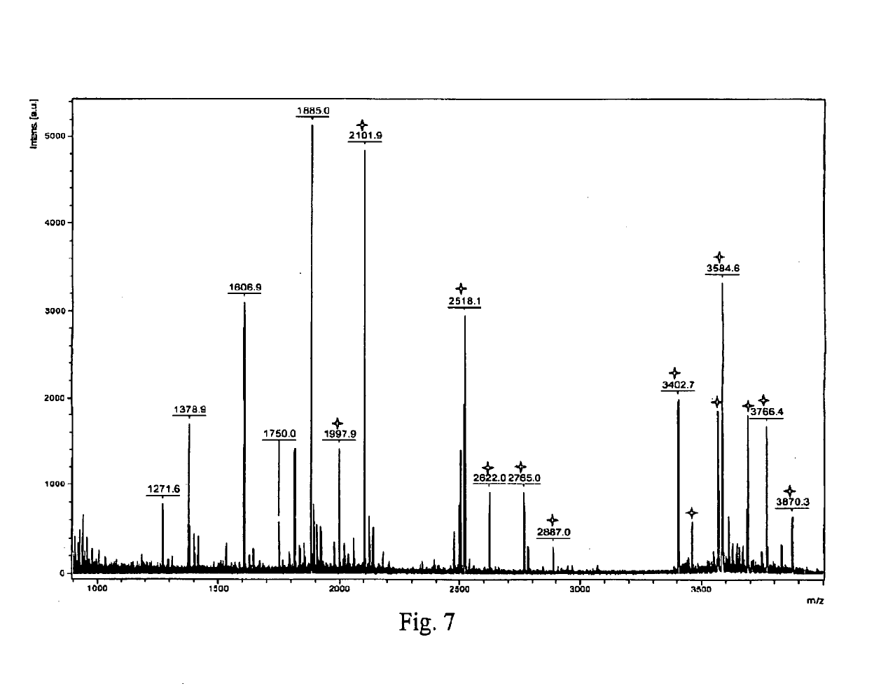

The same sample was analyzed a second time with neutral HCCE matrix (see FIG. 7).

In this case, 13 new intense signals can be clearly observed due to the detection discrimination effect of peptides cross-linked with compound JMV3378 and to the use of neutral HCCE matrix. Likewise, the intensity of the m/z=1885 signal corresponding to an unmodified peptide consequently decreased.

After having analyzed with the MSX-3D software the masses corresponding to the new signals observed, a prediction list was obtained and is presented in table 2 below.

TABLE 2
List of experimental and predicted masses
of peptides cross-linked by JMV3378.

| | | Devia- | | | |
|---|---|---|---|---|---|
| [M + H]⁺ | | tion | Sequence | Sequence | Modifi- |
| Experimental | Calculated | (ppm) | 1 | 2 | cations |
| 1997.89 | 1997.92 | 16 | 48-62 | | T1 |
| 2101.89 | 2101.96 | 33 | 1-16 | | T0 |
| 2518.08 | 2518.14 | 23 | 119-139 | | T0 |
| 2622.03 | 2622.39 | 136 | 99-118 | | T0 |
| 2765.04 | 2765.32 | 103 | 32-47 | 78-79 | T0 + T1 |
| 2886.96 | 2887.37 | 141 | 32-50 | | T0 |
| 3402.70 | 3402.78 | 23 | 48-56 | 63-79 | T0 + T2 |
| 3460.63 | 3462.64 | 581 | 57-63 | 80-98 | T0 + T2 |
| 3566.57 | 3566.74 | 46 | 46-56 | 134-147 | T0 + T2 |
| | 3566.93 | 101 | 57-78 | 97-102 | T0 + T2 |
| 3584.56 | 3583.96 | −168 | 63-79 | 140-153 | T2 |
| | 3584.77 | 57 | 43-62 | 140-147 | T2 |
| 3688.47 | 3688.84 | 99 | 48-56 | 63-79 | 2 × T0 + T2 |
| 3766.42 | 3766.08 | −89 | 46-50 | 78-102 | T2 |
| | 3766.70 | 74 | 46-62 | 140-147 | 2 × T0 + T2 |
| 3870.29 | 3869.87 | −106 | 78-98 | 146-153 | T0 + T2 |
| | 3870.02 | −68 | 63-79 | 140-153 | T0 + T2 |

The data provided by this analysis were verified with the crystalline structure of the protein. A total of seven cross-linking possibilities (types 1 and 2) consistent with the model crystalline structure of the protein were noted (see diagram 13).

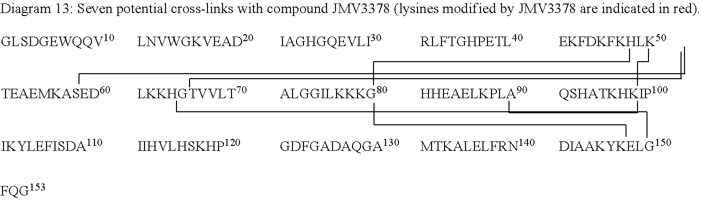

Moreover, according to the predictions, 16 lysines were modified with compound JMV3378. Lysines 16 and 118 were not considered accessible.

Identification by MALDI-TOF/TOF mass spectrometry of the cross-linked peptides is ongoing.

### Example 4

### Experimental Section

### 4-1 Analyses by MALDI-TOF Mass Spectrometry

The mass spectrometer used is a Bruker Daltonics Ultraflex. The spectrum acquisition and data analysis software are Flexcontrol and Flexanalysis version 2.2, respectively.

The spectrometer is equipped with a SCOUT source and desorption/ionization is carried out using a 337 nm wavelength nitrogen laser and the frequency used is 50 Hz. The power of the laser is adjustable via an attenuator.

### 4-1-1—Calibration

The MALDI-TOF spectrometry analyses were calibrated in external mode. Two calibration kits were used:
“Peptide calibration standard II” (Bruker #222570): 700 to 3200 Th
“Protein calibration standard I” (Bruker #206355): 5000 to 17500 Th

External calibration is carried out by depositing one of these commercial mixtures with a suitable matrix (e.g., α-cyano-4-hydroxycinnamic acid (HCCA) for “Peptide calibration standard II”, sinapinic acid (AS) for “Protein calibration standard I”) close to the sample(s) to analyze.

### 4-1-2—Sample Preparation

The samples are deposited on a Bruker Daltonics MTP 384-target polished-steel plate, according to two methods.

Dried Droplet Preparation

The dried droplet method was preferred in the majority of the analyses using HCCA matrix. It is carried out by co-crystallization of a droplet of matrix solution and a droplet of analyte solution. Each preparation can contain between 0.5 μl and 1 μl of each solution.

The solutions of HCCA matrix are prepared by saturation in a water/ACN/TFA solution (50:50:0.1).
Analyses with acidic HCCA matrix of post-enzymatic digestion protein modification tests:

0.5 μl (0.5 μmol) of 1 μM peptide solution in a water/ACN/TFA mixture (50:50:0.1) is co-crystallized with 0.5 μl of saturated acidic HCCA matrix in a water/ACN/TFA mixture (50:50:0.1).

Thin Layer Preparation

The thin layer method was used for each preparation using HCCE matrix. It consists in depositing 0.5 μl of saturated HCCE solution in acetone in a band on two labeled targets on the plate.

0.5 μl of analyte solution is deposited on a target containing the dried matrix preparation. Because of the difficulty of reproducing the appearance of the deposit by this method, it is preferable to renew the deposit a second time for each analysis.
Analyses with neutral HCCE matrix of post-enzymatic digestion protein modification tests:

0.5 μl (0.5 pmol) of 1 μM peptide solution in a water/ACN/TFA mixture (50:50:0.1) is co-crystallized on a preparation of 0.25 μl of saturated neutral HCCE matrix in acetone.

### 4-2 Synthesis of Model Peptides

Model peptides JMV3346, JMV3347, JMV3348, JMV3349 and JMV3350 were synthesized manually on a solid support according to an Fmoc strategy. Peptide 3 was synthesized on a solid support according to an Fmoc strategy using a microwave synthesizer.

The three peptides were synthesized with HBTU as coupling agent in the presence of NMM on an Fmoc-Rink Amide-aminomethyl-polystyrene resin (100-200 mesh, 0.7 mmol/g).

Synthesis of Peptide JMV3346

Three-letter code: H-Arg-Lys-Asn-Gly-Pro-Leu-Ile-Gly-Ala-Phe-NH₂

### Characterization of the Compound:

Appearance: white powder
Empirical formula: C₄₉H₈₂N₁₆O₁₁
Average molar mass=1071.28 g/mol
Average molar mass+3 TFA=1413.34 g/mol
Monoisotopic molar mass=1070.63 g/mol
Retention time (HPLC)=1.04 min
[M+2H]²⁺ₑₓₚ: m/z: 536.3 Th

Synthesis of Peptide JMV3347

Three-letter code: CCA-Arg-Lys-Asn-Gly-Pro-Leu-Ile-Gly-Ala-Phe-NH₂

### Characterization of the Compound:

Appearance: white powder
Empirical formula: C₅₉H₈₇N₁₇O₁₂
Average molar mass=1226.43 g/mol
Average molar mass+3 TFA=1454.47 g/mol
Monoisotopic molar mass=1225.67 g/mol
Retention time (HPLC)=1.26 min
[M+2H]²⁺ₑₓₚ: m/z: 613.9 Th

Synthesis of Peptide JMV3348

Three-letter code: HCCA-Arg-Lys-Asn-Gly-Pro-Leu-Ile-Gly-Ala-Phe-NH₂

### Characterization of the Compound:

Appearance: white powder
Empirical formula: C₅₉H₈₇N₁₇O₁₃
Average molar mass=1242.43 g/mol
Average molar mass+2 TFA=1470.47 g/mol
Monoisotopic molar mass=1241.67 g/mol
Retention time (HPLC)=1.17 min
[M+2H]²⁺ₑₓₚ: m/z: 621.9 Th

Synthesis of Peptide JMV3349:

Three-letter code: MCCA-Arg-Lys-Asn-Gly-Pro-Leu-Ile-Gly-Ala-Phe-NH₂

### Characterization of the Compound:

Appearance: white powder
Empirical formula: C₆₀H₈₉N₁₇O₁₃
Average molar mass=1256.45 g/mol
Average molar mass+2 TFA=1484.49 g/mol
Monoisotopic molar mass=1255.69 g/mol
Retention time (HPLC)=1.20 min
[M+2H]²⁺ₑₓₚ: m/z: 629.0 Th

Synthesis of Peptide JMV3350:

Three-letter code: ACCA-Arg-Lys-Asn-Gly-Pro-Leu-Ile-Gly-Ala-Phe-NH₂

### Characterization of the Compound:

Appearance: white powder
Empirical formula: C₆₁H₈₉N₁₇O₁₄
Average molar mass=1284.46 g/mol
Average molar mass+2 TFA=1512.50 g/mol
Monoisotopic molar mass=1283.68 g/mol
Retention time (HPLC)=1.26 min
[M+2H]²⁺ₑₓₚ: m/z: 643.1 Th

Synthesis of Peptide 3:

Three-letter code: H-Arg-Lys-Gly-Lys-Leu-Ala-Phe-NH₂

### Characterization of the Compound:

Appearance: white powder
Empirical formula: C₃₈H₆₇N₁₃O₇
Average molar mass=818.02 g/mol
Average molar mass+4 TFA=1274.10 g/mol
Monoisotopic molar mass=817.53 g/mol
Retention time (HPLC)=0.91 min
[M+H]⁺ₑₓₚ: m/z: 818.7 Da

### 4-3 Synthesis of Bifunctional Agents in Solution

4-3-1—Synthesis of N-hydroxysuccinimidyl N-(α-cyano-4-hydroxycinnamyl)iminodiacetate (JMV3378)

Synthesis of N′-(benzyloxycarbonyl)iminodiacetic acid (compound 1):

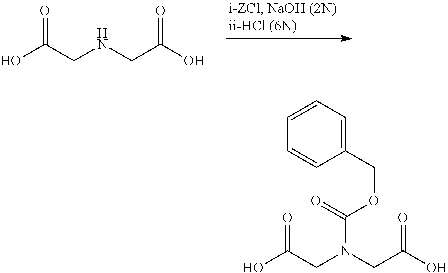

### Summary of Reagents:

| | MW | n | | m | v | d |
|---|---|---|---|---|---|---|
| Reagents | (g/mol) | (mmol) | eq | (g) | (ml) | (d/ml) |
| Iminodiacetic | 133.1 | 7.50 | 1.0 | 0.999 | — | — |
| acid | | | | | | |
| ZCl | 170.60 | 28.02 | 3.7 | 4.780 | 4.0 | 1.195 |

### Synthesis Protocol:

0.999 g of iminodiacetic acid is dissolved under magnetic stirring in 7.5 ml of 2 N aqueous soda solution. The pH of the medium is then greater than 10. The medium is placed in an, ice bath in order to stabilize its temperature at 0° C. Two times 2 ml of benzyl chloroformate are successively added. The pH of the medium is maintained at 10 by the addition of roughly 7 ml of 2 N aqueous soda solution.

The reaction is left stirring for two days and then the medium is extracted with 4 times 100 ml of diethyl ether. A stream of argon is set up in the medium which is then acidified with a 6 N aqueous HCl solution until a pH between 1 and 3 is reached. A white gel then appears.

The medium is then extracted with 4 times 100 ml of EA. The organic phases are combined and dried with MgSO₄ and then filtered on a fritted disc. The solvent is evaporated under reduced pressure.

2 g (28 mmol) of colorless oil is recovered.

### Characterization of the Compound:

Appearance: colorless oil

### Yield=100%

Empirical formula: C₁₂H₁₃NO₆
Average molar mass=267.23 g/mol
Monoisotopic molar mass=267.07 g/mol
Retention time (HPLC)=1.18 min
[M+H]⁺ₑₓₚ: m/z: 268.2 Da

### Rf(DCM/MeOH/AA-90:10:0.1; A B)=0.2

¹H NMR (300 MHz, CDCl₃, 300° K): δ(ppm) 4.07 (2H, s, CH₂—CO); 4.12 (2H, s, CH₂—CO); 5.11 (2H, s, CH₂-Ø); 7.27 (5H, s broad, phenyl); 7.86 (2H, s broad, CO₂H).

¹³NMR, Jmod (75 MHz, CDCl₃, 300° K): δ(ppm) 50.4 (CH₂—CO); 68.4 (CH₂—Ø); 127.8 (C₂₋₆ phenyl); 128.2 (C₄ phenyl); 128.5 (C_{3,5} phenyl); 135.5 (C₁ phenyl); 156.2 (N—CO); 173.2 (CO₂H).

Synthesis of tert-butyl N-(benzyloxycarbonyl)iminodiacetate (compound 2):

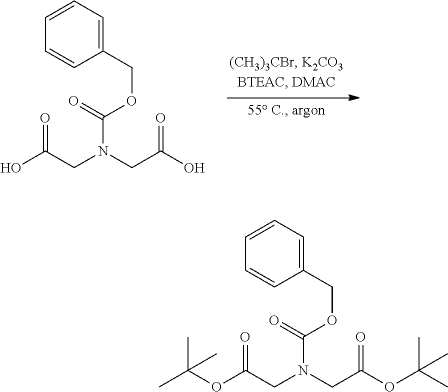

### Summary of Reagents:

| | MW | n | | m | v | d |
|---|---|---|---|---|---|---|
| Reagents | (g/mol) | (mmol) | eq | (g) | (ml) | (d/ml) |
| Compound 1 | 267.23 | 2.99 | 1.0 | 0.800 | — | — |
| Tert-butyl | 137.03 | 266.22 | 89.0 | 36.480 | 30.000 | 1.216 |
| bromide | | | | | | |
| K₂CO₃ | 138.00 | 72.46 | 24.2 | 10.000 | — | — |
| BTEAC | 227.78 | 7.20 | 2.4 | 1.640 | — | — |

### Synthesis Protocol:

0.800 g of compound 1 is dissolved in the presence of 1.640 g of benzyltriethylammonium chloride in 75 ml of DMAC under argon. 10 g of K₂CO₃ is added to the medium and then 30 ml of tert-butyl bromide is added.

The medium is left under rapid magnetic stirring for 30 hours at a temperature of 55° C. The medium is cooled in the open air up to room temperature.

After 15 days, the medium is placed in an ice bath and then 400 ml of cold water is added. The aqueous phase is extracted with 5 times 100 ml of EA. Each organic phase is then extracted with 3 times 50 ml of saturated aqueous NaHCO₃ solution and 2 times 50 ml of water. The combined organic phases are dried with MgSO₄, filtered on a fritted disc and then the solvent is evaporated under reduced pressure.

The compound is purified by column chromatography with DCM/MeOH/AA (98:2:0.1) as eluent.

193 mg (0.51 mmol) of colorless oil is recovered.

### Characterization of the Compound:

Appearance: colorless oil

### Yield=17%

Empirical formula: C₂₀H₂₉NO₆
Average molar mass=379.45 g/mol
Monoisotopic molar mass=379.20 g/mol
Retention time (HPLC)=1.99 min
[M+H]⁺ₑₓₚ: m/z: 380.1 Da

### Rf(DCM/MeOH/AA-90:10:0.1; A B)=0.86

¹H NMR (300 MHz, CDCl₃, 300° K): δ(ppm) 1.40 (9H, s, CH₃ tert-butyl); 1.46 (9H, s, CH₃ tert-butyl); 3.96 (2H, s, CH₂—CO); 4.05 (2H, s, CH₂—CO); 5.15 (2H, s, CH₂-Ø); 7.32 (5H, s broad, phenyl).

¹³C NMR, Jmod (75 MHz, CDCl₃, 300° K): δ(ppm) 27.9 (CH₃ tert-butyl); 28.1 (CH₃ tert-butyl); 49.9 (CH₂—CO); 50.2 (CH₂—CO); 67.7 (CH₂-Ø); 81.9 (C_{quaternary} tert-butyl); 127.7 (C_{2,6} phenyl); 127.9 (C₄ phenyl); 128.4 (C_{3,5} phenyl); 136.3 (C₁ phenyl); 168.5 (N—CO); 168.7 (CO₂tBu).

Synthesis of tert-butyl iminodiacetate (compound 4) from compound 2:

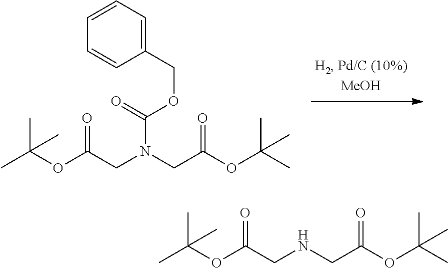

### Summary of Reagents:

| | MW | n | | m | v | d |
|---|---|---|---|---|---|---|
| Reagents | (g/mol) | (mmol) | eq | (g) | (ml) | (d/ml) |
| Compound 2 | 379.45 | 0.60 | 1.0 | 0.227 | — | — |

### Synthesis Protocol:

227 mg of compound 2 is dissolved in 60 ml of methanol. 30 mg of palladium on carbon is added. Hydrogen bubbling is set up in the medium. The reaction is left under magnetic stirring for 10 hours.

The flask is purged with argon. The medium is filtered on Celite and rinsed several times with methanol.

The solvent is then evaporated under reduced pressure and then the medium is taken up in 100 ml of EA and extracted with 2 times 50 ml of a saturated aqueous NaHCO₃ solution and then 2 times 50 ml of water. The organic phase is dried with MgSO₄ and filtered on a fritted disc. The solvent is then evaporated under reduced pressure.

73 mg (0.3 mmol) of colorless oil is recovered.

### Characterization of the Compound:

Appearance: colorless oil

### Yield=50%

Empirical formula: C₁₂H₂₃NO₄
Average molar mass=245.32 g/mol
Monoisotopic molar mass=245.16 g/mol
Retention time (HPLC)=1.09 min
[M+H]⁺ₑₓₚ: m/z: 246.4 Da

### Rf(AcOEt/EP-2:8; A B C)=0.37

¹H NMR (300 MHz, DMSO-d₆, 300° K): δ(ppm) 1.41 (9H, s, CH₃ tert-butyl); 1.42 (9H, s, CH₃ tert-butyl); 3.32 (4H, s, CH₂—CO).

¹³C NMR, Jmod (75 MHz, DMSO-d₆, 300° K): δ(ppm) 27.6 (CH₃ tert-butyl); 49.5 (CH₂—CO); 80.5 (C_{quaternary} tert-butyl); 170.1 (CO₂tBu).

Synthesis of tert-butyl N-benzyliminodiacetate (compound 3):

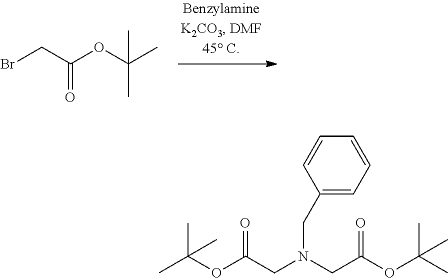

### Summary of Reagents:

| | MW | n | | m | v | d |
|---|---|---|---|---|---|---|
| Reagents | (g/mol) | (mmol) | eq | (g) | (ml) | (d/ml) |
| Tert-butyl | 195.06 | 35.75 | 3.0 | 6.975 | 5.280 | 1.321 |
| 2-bromoacetate | | | | | | |
| Benzylamine | 107.16 | 11.92 | 1.0 | 1.277 | 1.302 | 0.981 |
| K₂CO₃ | 138.00 | 64.13 | 5.4 | 8.850 | — | — |

### Synthesis Protocol:

5.280 ml of tert-butyl 2-bromoacetate is dissolved in 100 ml of DMF. 8.850 g of K₂CO₃ is added and the medium is placed under rapid magnetic stirring at a temperature of 45° C. 1.302 ml of benzylamine is added.

The reaction is quenched after 3 hours and the solvent is evaporated under reduced pressure. 400 ml of distilled water is added to the medium which is extracted with 4 times 250 ml of EA. Each organic phase is washed with 200 ml of water and 200 ml of brine. The organic phases are combined and dried with MgSO4 and then filtered on a fritted disc.

The compound is purified by column chromatography with PE/EA (95:5) as eluent.

3.990 g (11.89 mmol) of white powder is recovered.

### Characterization of the Compound:

Appearance: white powder

### Yield=99%

Empirical formula: C₁₉H₂₉NO₄
Average molar mass=335.44 g/mol
Monoisotopic molar mass=335.21 g/mol
Retention time (HPLC)=1.65 min
[M+H]⁺ₑₓₚ: m/z: 336.1 Da

### Rf(EA/PE-5:95; A B)=0.33

Melting point=45-50° C.

¹H NMR (300 MHz, CDCl₃, 300° K): δ(ppm) 1.44 (18H, s, CH₃ tert-butyl); 3.39 (4H, s, CH₂—CO); 3.88 (2H, s, CH₂-Ø); 7.31 (5H, m, phenyl).

¹³C NMR, Jmod (75 MHz, CDCl₃, 300° K): δ(ppm) 28.2 (CH₃ tert-butyl); 55.2 (CH₂—CO); 57.6 (CH₂-Ø); 80.9 (C_{quaternary} tert-butyl); 127.2 (C₄ phenyl); 128.8 (C_{3,5} phenyl); 129.1 (C_{2,6} phenyl); 138.6 (C₁ phenyl); 170.6 (CO).

Synthesis of Compound 4 from Compound 3:

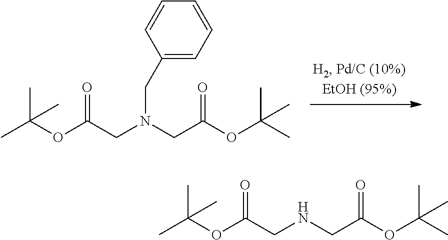

### Summary of Reagents:

| | MW | n | | m | v | d |
|---|---|---|---|---|---|---|
| Reagents | (g/mol) | (mmol) | eq | (g) | (ml) | (d/ml) |
| Compound 3 | 335.44 | 23.65 | 1.0 | 7.932 | — | — |

### Synthesis Protocol:

7.932 g of compound 3 is dissolved in 200 ml of 95% ethanol under magnetic stirring. 200 mg of palladium on carbon is introduced into the medium. Next, hydrogen bubbling is set up in the flask.

After 4 hours, the flask is purged with argon. The medium is filtered on Celite and rinsed with methanol. The solvent is then evaporated under reduced pressure.

5.771 g (23.52 mmol) of colorless oil is recovered.

### Characterization of the Compound:

Appearance: colorless oil

### Yield=99.5%

Empirical formula: C₁₂H₂₃NO₄
Average molar mass=245.32 g/mol
Monoisotopic molar mass=245.16 g/mol
Retention time (HPLC)=1.09 min
[M+H]⁺ₑₓₚ: m/z: 246.4 Da

### Rf(AcOEt/PE-2:8; A B C)=0.37

¹H NMR (300 MHz, DMSO-d₆, 300° K): δ(ppm) 1.41 (9H, s, CH₃ tert-butyl); 1.42 (9H, s, CH₃ tert-butyl); 3.32 (4H, s, CH₂—CO).

¹³C NMR, Jmod (75 MHz, DMSO-d₆, 300° K): δ(ppm) 27.6 (CH₃ tert-butyl) 49.5 (CH₂—CO); 80.5 (C_{quaternary} tert-butyl); 170.1 (CO₂tBu).

Synthesis of tert-butyl N-(2-chloroacetyl)iminodiacetate (compound 5):

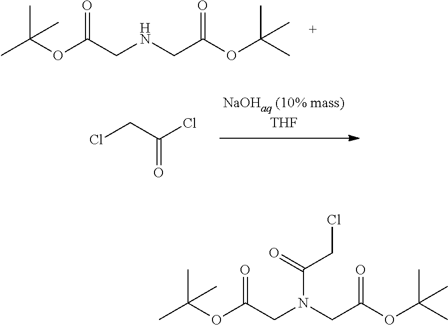

### Summary of Reagents:

| | MW | n | | m | v | d |
|---|---|---|---|---|---|---|
| Reagents | (g/mol) | (mmol) | eq | (g) | (ml) | (d/ml) |
| Compound 5 | 245.32 | 5.01 | 1.0 | 1.230 | — | — |
| 2-Chloroacetyl | 112.94 | 25.07 | 5.0 | 2.832 | 1.997 | 1.418 |
| chloride | | | | | | |

### Synthesis Protocol:

1.230 g of compound 5 is dissolved in 40 ml of THF and 3 ml of 2.5 M aqueous soda solution is added. The medium is placed under rapid magnetic stirring. 1.997 ml of 2-chloroacetyl chloride is added slowly and of 1 N soda is regularly added to maintain the pH of the solution below 8.

After 5 minutes of stirring, the THF is evaporated under reduced pressure. 150 ml of EA is added to the medium which is then extracted with 4 times 100 ml of distilled water. The organic phase is then dried with MgSO₄, filtered on a fritted disc and the solvent is evaporated under reduced pressure.

1.605 g (4.99 mmol) of colorless oil is recovered.

### Characterization of the Compound:

Appearance: colorless oil

### Yield=99.5%

Empirical formula: C₁₄H₂₄ClNO₅
Average molar mass=321.80 g/mol
Monoisotopic molar mass=321.13 g/mol
Retention time (HPLC)=1.78 min
[M+H]⁺ₑₓₚ: m/z: 322.4 Da

### Rf(EA/PE-3:7; A B)=0.47

¹H NMR (300 MHz, CDCl₃, 300° K): δ(ppm) 1.44 (9H, s, CH₃ tert-butyl); 1.47 (9H, s, CH₃ tert-butyl); 4.05 (2H, s, CH₂—CO); 4.06 (2H, s, CH₂—CO); 4.07 (2H, s, CH₂Cl).

¹³C NMR, Jmod (75 MHz, CDCl₃, 300° K): δ(ppm) 27.9 (CH₃ tert-butyl); 27.9 (CH₃ tert-butyl); 40.7 (CH₂Cl); 49.2 (CH₂—CO); 51.7 (CH₂—CO); 82.2 (C_{quaternary} tert-butyl); 82.9 (C_{quaternary} tert-butyl); 166.9 (N—CO); 167.5 (CO₂tBu); 167.7 (CO₂tBu).

Synthesis of tert-butyl N-(2-cyanoacetyl)iminodiacetate (compound 6):

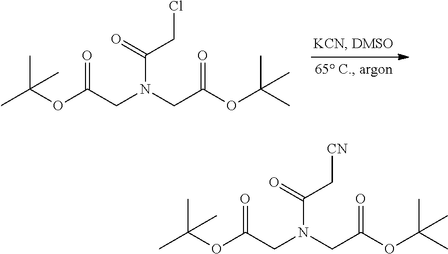

### Summary of Reagents:

| | MW | n | | m | v | d |
|---|---|---|---|---|---|---|
| Reagents | (g/mol) | (mmol) | eq | (g) | (ml) | (d/ml) |
| Compound 5 | 321.80 | 5.76 | 1.0 | 1.854 | — | — |
| KCN | 65.00 | 6.34 | 1.1 | 0.412 | — | — |

### Synthesis Protocol:

1.854 g of compound 5 is dissolved in 80 ml of DMSO under argon and magnetic stirring. 412 mg of potassium cyanide is added to the medium which is placed at a temperature of 65° C.

The reaction is quenched after 1 hour by the addition of 400 ml of water. The solvent is then evaporated under reduced pressure. The medium is taken up in 200 ml of water and then extracted with 4 times 200 ml of EA. Each organic phase is washed with 250 ml of brine. The organic phases are combined and then dried with MgSO₄ and filtered on a fritted disc. The solvent is evaporated under reduced pressure.

The crude reaction product is purified by column chromatography with an EA/PE mixture (3:7) as eluent.

1.355 g (4.34 mmol) of colorless oil is recovered.

### Characterization of the Compound:

Appearance: colorless oil

### Yield=76%

Empirical formula: C₁₅H₂₄N₂O₅
Average molar mass=312.36 g/mol
Monoisotopic molar mass=312.17 g/mol
Retention time (HPLC)=1.66 min
[M+H]⁺ₑₓₚ: m/z: 313.5 Da

### Rf(EA/PE-3:7; A B)=0.37

¹H NMR (300 MHz, CDCl₃, 300° K): δ(ppm) 1.45 (9H, s, CH₃ tert-butyl); 1.48 (9H, s, CH₃ tert-butyl); 3.55 (2H, s, CH₂—CN); 3.97 (2H, s, CH₂—CO); 4.06 (2H, s, CH₂—CO).

¹³C NMR, Jmod (75 MHz, CDCl₃, 300° K): δ(ppm) 24.7 (CH₂—CN); 27.9 (CH₃ tert-butyl); 27.9 (CH₃ tert-butyl); 49.5 (CH₂—CO); 51.5 (CH₂—CO); 82.6 (C_{quaternary} tert-butyl); 83.6 (C_{quaternary} tert-butyl); 162.6 (N—CO); 166.9 (CO₂tBu); 167.4 (CO₂tBu).

Synthesis of tert-butyl N-(α-cyano-4-hydroxycinnamyl)iminodiacetate (compound 7):

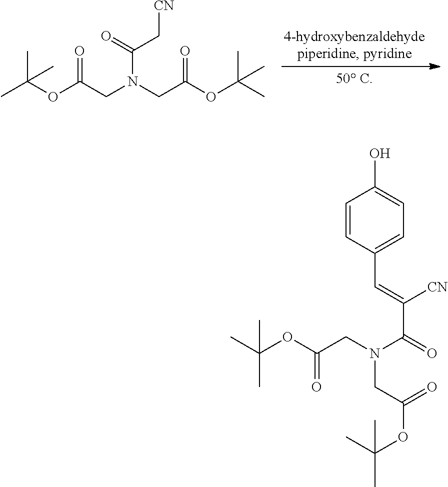

### Summary of Reagents:

| | MW | n | | m | v | d |
|---|---|---|---|---|---|---|
| Reagents | (g/mol) | (mmol) | eq | (g) | (ml) | (d/ml) |
| Compound 6 | 312.36 | 0.64 | 1.0 | 0.199 | — | — |
| 4-Hydroxy- | 122.12 | 0.64 | 1.0 | 0.078 | — | — |
| benzaldehyde | | | | | | |
| Pyridine | 79.10 | 83.20 | 130.0 | 6.581 | 6.729 | 0.978 |
| Piperidine | 85.15 | 0.32 | 0.5 | 0.028 | 0.032 | 0.861 |

### Synthesis Protocol:

199 mg of compound 6 is dissolved in 6.729 ml of pyridine and 32 μl of piperidine. 78 mg of 4-hydroxybenzaldehyde is added to the medium under magnetic stirring at 50° C. After 15 hours, the solvent is evaporated under reduced pressure. The medium is then taken up in 100 ml of EA and then extracted with 4 times 100 ml of 1 M aqueous KHSO₄ solution and 3 times 50 ml of saturated aqueous sodium sulfite solution acidified with a few milliliters of acetic acid. A final washing is carried out with 3 times 100 ml of brine. The organic phase is dried with MgSO₄, filtered on a fritted disc and the solvent is evaporated under reduced pressure.

258 mg (0.47 mmol) of yellow powder is recovered.

### Characterization of the Compound:

Appearance: yellow powder

### Yield=74%

Empirical formula: C₂₂H₂₈N₂O₆
Average molar mass=416.47 g/mol
Monoisotopic molar mass=416.19 g/mol
Retention time (HPLC)=1.85 min

[M+H]⁺ₑₓₚ: m/z: 417.38 Da

### Rf(CHCl₃/MeOH/AA-120:10:5; A B)=0.43

Melting point=57-59° C.

¹H NMR (300 MHz, CDCl₃, 300° K): δ(ppm) 1.49 (9H, s, CH₃ tert-butyl); 1.50 (9H, s, CH₃ tert-butyl); 4.16 (2H, s, CH₂—CO); 4.21 (2H, s, CH₂—CO); 6.64 (2H, d, Jₒ=9 Hz, H_{6,8} cinnamyl); 7.20 (2H, d, Jₒ=9 Hz, H_{5,9} cinnamyl); 7.67 (1H, s, H₃ cinnamyl).

¹³C NMR, Jmod (75 MHz, CDCl₃, 300° K): δ(ppm) 27.9 (CH₃ tert-butyl); 28.1 (CH₃ tert-butyl); 48.7 (CH₂—CO); 51.5 (CH₂—CO); 82.8 (C_{quaternary} tert-butyl); 102.2 (C₂ cinnamyl); 116.3 (C_{6,8} cinnamyl); 116.6 (CN); 123.9 (C₄ cinnamyl); 132.6 (C_{5,9} cinnamyl); 149.8 (C₃ cinnamyl); 159.9 (C₇ cinnamyl); 164.5 (C₁ cinnamyl); 166.8 (CO₂tBu); 167.4 (CO₂tBu).

Synthesis of N′-(α-cyano-4-hydroxycinnamyl)iminodiacetic acid (compound 8):

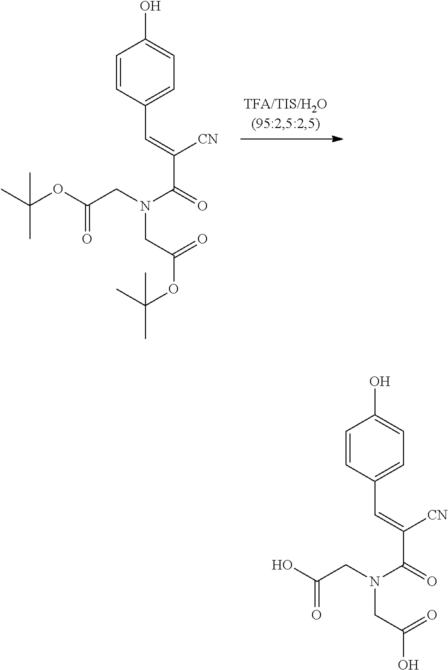

### Summary of Reagents:

| | MW | n | | m | v | d |
|---|---|---|---|---|---|---|
| Reagents | (g/mol) | (mmol) | eq | (g) | (ml) | (d/ml) |
| Compound 7 | 416.47 | 0.60 | 1.0 | 0.250 | — | — |

### Synthesis Protocol:

250 mg of compound 7 is dissolved in 50 ml of a TFA/TIS/H₂O mixture (95:2.5:2.5) under magnetic stirring. After 1 hour, the solvent is evaporated under reduced pressure. The medium is taken up in 50 ml of a water/ACN mixture (50:50), frozen with liquid nitrogen and lyophilized.

150 mg (0.49 mmol) of pale yellow powder is recovered.

### Characterization of the Compound:

Appearance: pale yellow powder

### Yield=82%

Empirical formula: C₁₄H₁₂N₂O₆
Average molar mass=304.26 g/mol
Monoisotopic molar mass=304.07 g/mol
Retention time (HPLC)=0.89 min
[M+H]⁺ₑₓₚ: m/z: 305.1 Da

### Rf(CHCl₃/MeOH/AA-85:10:5; A B)=0.47

Melting point=206° C.

¹H NMR (300 MHz, DMSO-d₆, 300° K): δ(ppm) 4.07 (2H, s, CH₂—CO); 4.34 (2H, s, CH₂—CO); 6.92 (2H, d, Jₒ=9 Hz, H_{6,8} cinnamyl); 7.64 (1H, s, H₃ cinnamyl); 7.83 (2H, d, Jₒ=9 Hz, H_{5,9} cinnamyl).

¹³C NMR, Jmod (75 MHz, DMSO-d₆, 300° K): δ(ppm) 48.7 (CH₂—CO); 51.1 (CH₂—CO); 100.0 (C₂ cinnamyl); 116.1 (C_{6,8} cinnamyl); 116.4 (CN); 123.1 (C₄ cinnamyl); 132.5 (C_{5,9} cinnamyl); 150.9 (C₃ cinnamyl); 161.6 (C₇ cinnamyl); 164.8 (C₁ cinnamyl); 169.8 (CO₂H).

Synthesis of N-hydroxysuccinimidyl N-(α-cyano-4-hydroxycinnamyl)iminodiacetate (JMV3378):

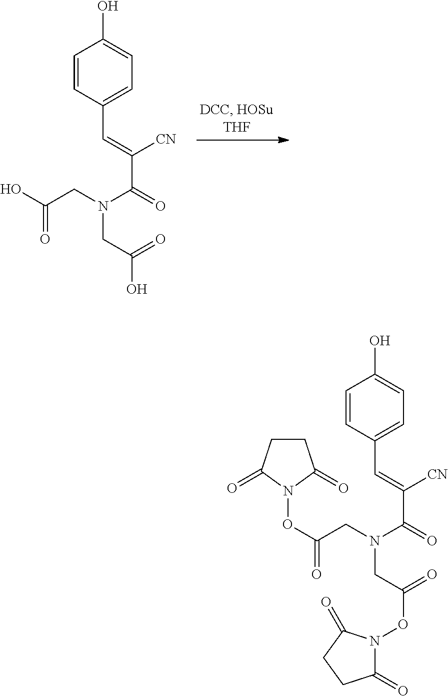

### Summary of Reagents:

| | MW | n | | m | v | d |
|---|---|---|---|---|---|---|
| Reagents | (g/mol) | (mmol) | eq | (g) | (ml) | (d/ml) |
| Compound 8 | 304.25 | 2.48 | 1.0 | 0.755 | — | — |
| DCC | 206.30 | 10.25 | 4.1 | 2.115 | — | — |
| HOSu | 115.09 | 6.21 | 2.5 | 0.715 | — | — |

### Synthesis Protocol:

775 mg of compound 8 is dissolved in 100 ml of THF and 2.115 g of DCC is added. After 10 minutes, the medium becomes cloudy and 715 mg of HOSu is added.

After 1 hour, the solvent is evaporated under reduced pressure and the medium is taken up in a minimum of cold DMF and placed in an ice bath for 10 minutes. Dicyclohexylurea precipitates as white powder. The medium is filtered cold on a fritted disc with a porosity of 4. The process is repeated a second time. The solvent is then evaporated under reduced pressure. The precipitate is dissolved in 300 ml of EA and extracted with 2 times 100 ml of 1 M aqueous KHSO₄ solution. The organic phase is dried with MgSO₄, filtered on a fritted disc and the solvent is evaporated under reduced pressure.

0.850 g (1.71 mmol) of pale yellow powder is recovered.

### Characterization of the Compound:

Appearance: pale yellow powder

### Yield=69%

Empirical formula: C₂₂H₁₈N₄O₁₀
Average molar mass=498.40 g/mol
Monoisotopic molar mass=498.10 g/mol
Retention time (HPLC)=1.27 min
[M+H]⁺ₑₓₚ: m/z: 499.1 Da

### Rf(DCM/MeOH-9:1; A B)=0.45

¹H NMR (300 MHz, DMSO-d₆, 300° K): δ(ppm) 2.82 (8H, s, CH₂ succinimidyl); 3.30 (4H, s, N—CH₂—CO); 6.91 (2H, d, Jₒ=9 Hz, H_{6,8} cinnamyl); 7.59 (1H, s, H₃ cinnamyl); 7.87 (2H, d, Jₒ=9 Hz, H_{5,9} cinnamyl); 10.56 (1H, s, OH).

¹³C NMR, Jmod (75 MHz, DMSO-d₆, 300° K): δ(ppm) 25.7 (CH₂ succinimidyl); 25.9 (CH₂ succinimidyl); 60.2 (CH₂—CO); 99.6 (C₂ cinnamyl); 116.5 (C_{6,8} cinnamyl); 116.7 (CN); 122.8 (C₄ cinnamyl); 133.3 (C_{5,9} cinnamyl); 151.0 (C₃ cinnamyl); 162.4 (C₇ cinnamyl); 165.6 (C₁ cinnamyl); 170.0 (CO succinimidyl); 170.2 (CH₂—CO).

4-3-2—Synthesis of N-hydroxysuccinimidyl (S)-3-(4-benzoylphenyl)-2-(α-cyano-4-hydroxycinnamido)propanoate (JMV3480)

Synthesis of (S)-3-(4-benzoylphenyl)-2-(α-cyano-4-hydroxycinnamido)propanoic acid (compound 12):

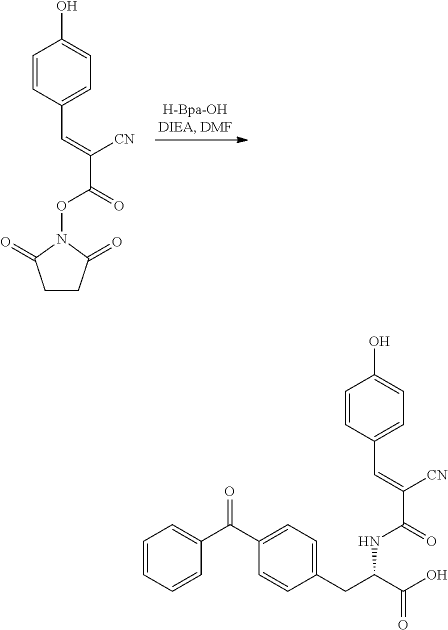

### Summary of Reagents:

| | MW | n | | m | v | d |
|---|---|---|---|---|---|---|
| Reagents | (g/mol) | (mmol) | eq | (g) | (ml) | (d/ml) |
| JMV3155 | 286.25 | 0.75 | 1.1 | 0.215 | — | — |
| H-Bpa-OH | 269.30 | 0.68 | 1.0 | 0.183 | — | — |
| DIEA | 129.25 | 0.82 | 1.2 | 0.106 | 1.140 | 0.755 |

### Synthesis Protocol:

183 mg of 4-benzoyl-L-phenylalanine acid (H-Bpa-OH) is dissolved in 15 ml of DMF under magnetic stirring after the addition of 140 μl of DIEA. After 5 minutes, 215 mg of JMV3155 is added to the medium.

After 24 hours, the solvent is evaporated under reduced pressure. The medium is taken up in 200 ml of EA and then extracted with 3 times 80 ml of 1 M aqueous KHSO₄ solution. The organic phase is then dried with MgSO₄ and then filtered on a fritted disc. The solvent is evaporated under reduced pressure.

The compound is purified by preparative HPLC with the following gradient:
0%→20% ACN in 2 min;
20%→50% ACN in 30 min.

The fractions containing the purified compound are combined, frozen with liquid nitrogen and lyophilized.

156 mg (0.36 mmol) of yellow powder is recovered.

### Characterization of the Compound:

Appearance: yellow powder

### Yield=52%

Empirical formula: C₂₆H₂₀N₂O₅
Average molar mass=440.45 g/mol
Monoisotopic molar mass=440.14 g/mol
Retention time (HPLC)=1.53 min
[M+H]⁺ₑₓₚ: m/z: 441.0 Da

### Rf(CHCl₃/MeOH/AA-85:10:5; A B)=0.47

¹H NMR (300 MHz, DMSO-d₆, 300° K): δ(ppm) 3.28-3.36 (2H, m, H_{β}); 4.61-4.68 (1H, m, H_{α}); 6.67 (2H, d, Jₒ=9 Hz, H_{6,8} cinnamyl); 6.96 (2H, d, Jₒ=9 Hz, H_{5,9} cinnamyl); 7.47 (2H, d, Jₒ=9 Hz, H_{3,5} p-methylbenzoyl); 7.63-7.70 (5H, m, H_{2,6} p-methylbenzoyl and H_{3,4,5} benzoyl); 7.86 (2H, d, Jₒ=9 Hz, H_{2,6} benzoyl); 7.87 (1H, s, H₃ benzoyl); 8.51 (1H, s, OH).

¹³C NMR, Jmod (75 MHz, DMSO-d₆, 300° K): δ(ppm) 36.1 (C_{β}); 53.9 (C_{α}); 100.7 (C₂ cinnamyl); 116.2 (C_{6,8} cinnamyl); 116.9 (CN); 122.7 (C₄ cinnamyl); 128.5 (C_{5,9} cinnamyl); 129.3 (C_{3,5} p-methylbenzoyl); 129.4 (C_{3,5} benzoyl); 129.6 (C p-methylbenzoyl); 132.5 (C₄ benzoyl); 132.9 (C_{2,6} benzoyl); 135.2 (C₁ p-methylbenzoyl); 137.2 (C₁ benzoyl); 143.1 (C₄ p-methylbenzoyl); 150.7 (C₃ cinnamyl); 161.7 (C₇ p-cinnamyl); 161.9 (C₁ cinnamyl); 172.2 (CO₂H); 195.4 (CO-Ø).

Synthesis of JMV3480:

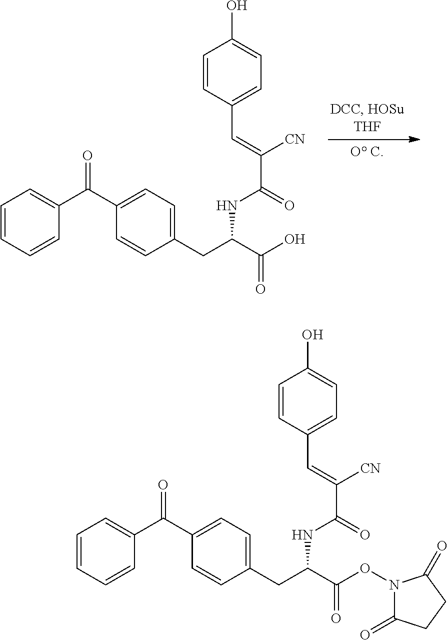

### Summary of Reagents:

| | MW | n | | m | v | d |
|---|---|---|---|---|---|---|
| Reagents | (g/mol) | (mmol) | eq | (g) | (ml) | (d/ml) |
| Compound 12 | 440.45 | 0.12 | 1.0 | 0.052 | — | — |
| DCC | 206.30 | 0.28 | 2.3 | 0.058 | — | — |
| HOSu | 115.09 | 0.29 | 2.4 | 0.033 | — | — |

### Synthesis Protocol:

52 mg of compound 12 is dissolved in 5 ml of THF and 58 mg of DCC is added under magnetic stirring. The temperature of the medium is lowered to 0° C. and then 33 mg of HOSu is added.

After 5 hours, dicyclohexylurea precipitated as white powder is filtered cold on a fritted disc with a porosity of 4. The solvent is then evaporated under reduced pressure and then the medium is taken up in 150 ml of EA and extracted with 4 times 100 ml of 1 M aqueous KHSO₄ solution. The organic phase is dried with MgSO₄, filtered on a fritted disc and the solvent is evaporated under reduced pressure.

60 mg (0.11 mmol) of yellow powder is recovered.

### Characterization of the Compound:

Appearance: yellow powder

### Yield=93%

Empirical formula: C₃₀H₂₃N₃O₇
Average molar mass=537.52 g/mol
Monoisotopic molar mass=537.15 g/mol
Retention time (HPLC)=1.69 min
[M+H]⁺ₑₓₚ: m/z: 538.2 Da

### Rf(CHCl₃/MeOH/AA-85:10:5; A B)=0.7

4-3-3 Synthesis of 5-(α-cyano-4-hydroxycinnamido)isophthalic acid (compound 11)

Synthesis of 5-(2-chloroacetamido)isophthalic acid (compound 9):

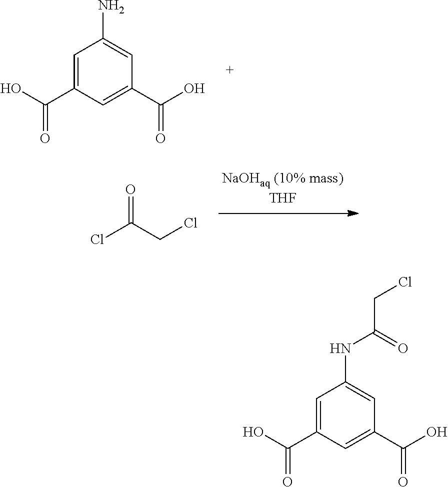

### Summary of Reagents:

| | MW | n | | m | v | d |
|---|---|---|---|---|---|---|
| Reagents | (g/mol) | (mmol) | eq | (g) | (ml) | (d/ml) |
| 5-Amino- | 181.15 | 19.41 | 1.0 | 3.516 | — | — |
| isophthalic acid | | | | | | |
| 2-Chloroacetyl | 112.94 | 97.04 | 5.0 | 10.960 | 7.729 | 1.418 |
| chloride | | | | | | |

### Synthesis Protocol:

3.516 g of 5-aminoisophthalic acid is dissolved in 150 ml of THF containing 20 ml of 10% mass aqueous soda solution. After 5 minutes under magnetic stirring, the diacid is completely solubilized. 7.729 ml of 2-chloroacetyl chloride is added slowly to the medium and the pH of the solution is maintained at 10 by the addition of 1 N soda solution under rapid magnetic stirring. The reaction is exothermic. After 5 minutes of stirring, the THF is evaporated under reduced pressure.

The medium is taken up in 400 ml of EA and then extracted with 2 times 100 ml of 1 M aqueous KHSO₄ solution. The aqueous phases are extracted with 2 times 100 ml of EA. The organic phases are combined and then dried with MgSO₄ and filtered on a fritted disc. The medium is concentrated until the solution is saturated (approximately 300 ml). The compound is then precipitated by the addition of 300 ml of PE. The solid is filtered on a fritted disc with a porosity of 4.

4.762 g (18.49 mmol) of white powder is recovered.

### Characterization of the Compound:

Appearance: white powder

### Yield=96%

Empirical formula: C₁₀H₈ClNO₅
Average molar mass=257.63 g/mol
Monoisotopic molar mass=257.01 g/mol
Retention time (HPLC)=0.95 min
[M+H]⁺ₑₓₚ: m/z: 258.0 Da

### Rf(CHCl₃/MeOH/AA-40:10:5; A B)=0.63

¹H NMR (300 MHz, DMSO-d₆, 300° K): δ(ppm) 4.29 (2H, s, CH₂Cl); 8.20 (1H, s, H₄ 5-aminoisophthalic); 8.43 (2H, S, H_{2,6} 5-aminoisophthalic); 10.66 (2H, s, CO₂H).

¹³C NMR, Jmod (75 MHz, DMSO-d₆, 300° K): δ(ppm) 43.5 (CH₂Cl); 123.7 (O_{2,6} 5-aminoisophthalic); 125.1 (C₄ 5-aminoisophthalic); 131.9 (C_{3,5} 5-aminoisophthalic); 139.1 (C₁ 5-aminoisophthalic); 165.2 (NH—CO); 166.3 (CO₂H).

Synthesis of 5-(2-cyanoacetamido)isophthalic acid (compound 10):

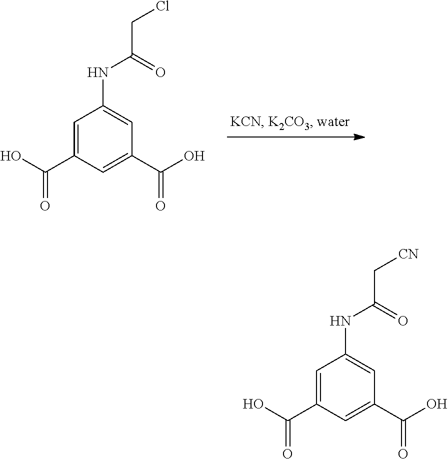

### Summary of Reagents:

| | MW | n | | m | v | d |
|---|---|---|---|---|---|---|
| Reagents | (g/mol) | (mmol) | eq | (g) | (ml) | (d/ml) |
| Compound 9 | 257.63 | 18.48 | 1.0 | 4.762 | — | — |
| KCN | 65.00 | 74.34 | 4.0 | 4.832 | — | — |
| K₂CO₃ | 138.00 | 27.51 | 1.5 | 3.796 | — | — |

### Synthesis Protocol:

4.762 g of compound 9 is placed in 150 ml of distilled water and 3.796 g of K₂CO₃ is added under rapid magnetic stirring. The solution becomes homogeneous after 5 minutes of stirring. 4.832 g of potassium cyanide is added slowly. The solution quickly turns red.

After 2 days, the reaction is quenched and the medium is acidified by the addition of a few milliliters of 6 N aqueous HCl solution until a pH below 3 is reached. The medium is then extracted with 8 times 100 ml of EA. Each organic phase is washed with 100 ml 1 M aqueous KHSO₄ solution. The organic phases are combined and dried with MgSO₄ and are filtered on a fritted disc. The solvent is evaporated under reduced pressure.

3.511 g (14.15 mmol) of white powder is recovered.

### Characterization of the Compound:

Appearance: white powder

### Yield=77%

Empirical formula: C₁₁H₈N₂O₅
Average molar mass=248.19 g/mol
Monoisotopic molar mass=248.04 g/mol
Retention time (HPLC)=0.83 min
[M+H]⁺ₑₓₚ: m/z: 249.2 Da

### Rf(CHCl₃/MeOH/AA-40:10:5; A B)=0.30

¹H NMR (300 MHz, DMSO-d₆, 300° K): δ(ppm) 3.94 (2H, s, CH₂—CN); 8.20 (1H, s, H₄ 5-aminoisophthalic); 8.38 (2H, s, H_{2,6} 5-aminoisophthalic); 10.67 (2H, s, CO₂H).

¹³C NMR, Jmod (75 MHz, DMSO-d₆, 300° K): δ(ppm) 26.8 (CH₂Cl); 115.6 (CN); 123.6 (C_{2,6} 5-aminoisophthalic); 152.1 (C₄ 5-aminoisophthalic); 131.9 (C_{3,5} 5-aminoisophthalic); 138.9 (C, 5-aminoisophthalic); 161.6 (NH—CO); 166.3 (CO₂H).

Synthesis of 5-(α-cyano-4-hydroxycinnamido)isophthalic acid (compound 11):

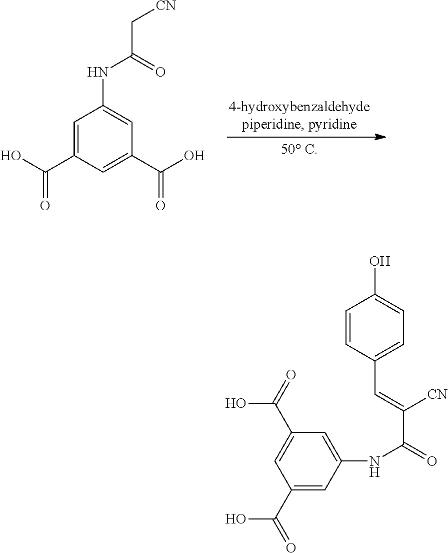

### Summary of Reagents:

| | MW | n | | m | v | d |
|---|---|---|---|---|---|---|
| Reagents | (g/mol) | (mmol) | eq | (g) | (ml) | (d/ml) |
| Compound 10 | 248.19 | 2.01 | 1.0 | 0.500 | — | — |
| 4-Hydroxy- | 122.12 | 2.01 | 1.0 | 0.246 | — | — |
| benzaldehyde | | | | | | |
| Piperidine | 85.15 | 1.01 | 0.5 | 0.086 | 0.100 | 0.861 |
| Pyridine | 79.10 | 262.10 | 130.4 | 20.732 | 21.198 | 0.978 |

### Synthesis Protocol:

500 mg of compound 10 is dissolved in 200 ml of pyridine and 100 μl of piperidine is added. After 5 minutes, 246 mg of 4-hydroxybenzaldehyde is added and the medium is placed in an oil bath at 50° C. for 3 hours.

The medium is cooled slowly to room temperature. A 1 M aqueous KHSO₄ solution is added drop by drop. The pH is below 4. A yellow precipitate appears and the solution is crushed with a spatula. The precipitate is filtered on a fritted disc with a porosity of 4 and then dissolved in DMF. The solvent is evaporated under reduced pressure. The solid is taken up in 150 ml of 1 M aqueous KHSO₄ solution and then crushed and filtered as before. The solid is then dissolved in a 50% ACN/water mixture, frozen in liquid nitrogen and then lyophilized.

700 mg (1.99 mmol) of yellow powder is recovered.

### Characterization of the Compound:

Appearance: yellow powder

### Yield=99%

Empirical formula: C₁₈H₁₂N₂O₆
Average molar mass=352.30 g/mol
Monoisotopic molar mass=352.07 g/mol
Retention time (HPLC)=1.30 min
[M+H]⁺ₑₓₚ: m/z: 353.0 Da

### Rf(CHCl₃/MeOH/AA-40:10:5; A B)=0.6

¹H NMR (300 MHz, DMSO-d₆, 300° K): δ(ppm) 6.98 (2H, d, Jₒ=9 Hz, H_{6,8} cinnamyl); 7.94 (2H, d, Jₒ=9 Hz, H_{5,9} cinnamyl); 8.23 (2H, s, H_{2,6} 5-aminoisophthalic); 8.55 (1H, s, H₄ 5-aminoisophthalic); 8.55 (1H, s, H₃ cinnamyl); 10.60 (2H, s, CO₂H).

¹³C NMR, Jmod (75 MHz, DMSO-d₆, 300° K): δ(ppm) 101.8 (C₂ cinnamyl); 116.4 (C_{6,8} cinnamyl); 116.8 (CN); 122.8 (C₄ cinnamyl); 124.9 (C₂₋₆ 5-aminoisophthalic); 125.3 (C₄ 5-aminoisophthalic); 131.7 (C_{3,5} 5-aminoisophthalic); 133.1 (C_{5,9} cinnamyl); 139.2 (C₁ 5-aminoisophthalic); 151.1 (C₃ cinnamyl); 161.6 (C₇ cinnamyl); 162.1 (C₁ cinnamyl); 166.4 (CO₂H).

1.-13. (canceled)

14. A protein cross-linking agent of formula (I) 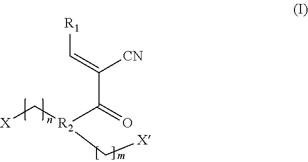
wherein
R₁ is an aryl group optionally substituted one or more times by a group selected from the group consisting of hydroxy, C₁-C₄ alkyl, OBoc, SO₃Na, Deu, C₁-C₄ alkoxy,
R₂ is N, 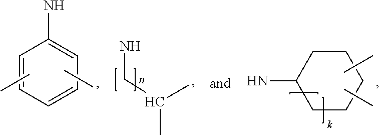
n and m are identical or different integers between 0 and 10, preferably between 1 and 5,
p is an integer between 0 and 5,
k is 0, 1, 2 or 3,
X and X′ are identical or different and are a protein reactive functional group.

15. The cross-linking agent of claim 14, wherein n=m.

16. The cross-linking agent of claim 14, wherein R₁ is a phenoxy group.

17. The cross-linking agent of claim 14, wherein R₂ is

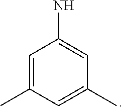

18. The cross-linking agent of claim 14, wherein X and X′ are identical or different and are selected from the group consisting of imidoester, N-hydroxysuccinimide ester, isocyanate, isothiocyanate, N-maleimide, disulfide, 1,2-dicarbonyl, benzophenone and aryl azide functional groups.

19. The cross-linking agent of claim 14, of formula (II)

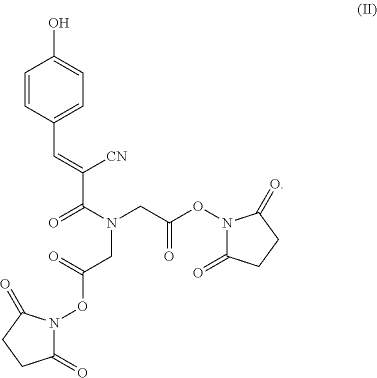

20. The cross-linking agent of claim 14, of formula (III)

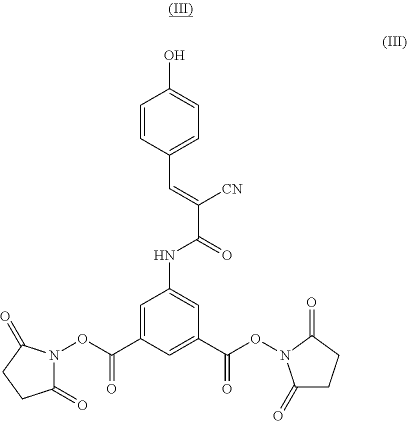

21. The cross-linking agent of claim 14, of formula (IV)

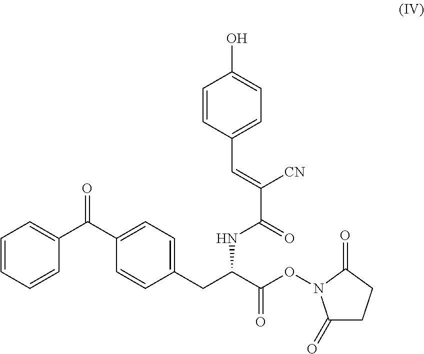

22. A method for preparing the cross-linking agent of claim 14, comprising the following steps:
(a) peptide coupling between the amine RR′NH and the carboxylic acid 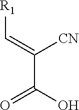
whose acid functional group is optionally activated,
to give 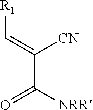
with R₁ as previously defined,
with R and R′:
each representing -(alkyl)-COOY, or
one representing H and the other 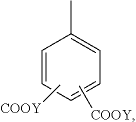
or
one representing H and the other 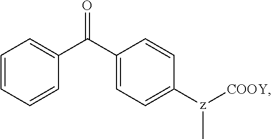
with Y representing H or tBu,
and with Z representing a C₁-C₈ alkyl group;
or (a′)
(i) reaction of RR′NH with 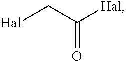
where Hal is a halogen atom,
in the presence of a base to give 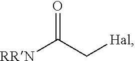
(ii) reaction of 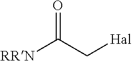
with a cyanide, such as KCN, to give 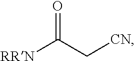
(iii) reaction of 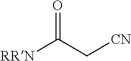
with R₁CHO in the presence of a base to give 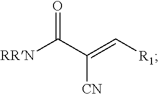
(b) optional hydrolysis of the ester obtained in step (a) or (a′) in acid to give 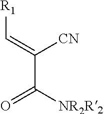
with R₂ and R₂′:
each representing -(alkyl)-COOH, or
one representing H and the other 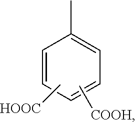
or
one representing H and the other 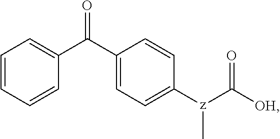
with Z representing a C₁-C₈ alkyl group;
(c) reaction of the compound obtained in preceding step (b) with a protein reactive functional group.

23. A method for the mass spectrometry analysis of the three-dimensional structure of a protein comprising the use of the cross-linking agent of claim 14.

24. A method for the structural analysis of a protein or of a protein complex comprising the following steps:
a) cross-linking of the protein or of the protein complex on the cross-linking agent according to claim 14 by the X and/or X′ functional groups,
b) enzymatic digestion of the protein or protein complex bound to the cross-linking agent according to claim 14,
c) analysis by mass spectrometry.

25. The method of claim 24, wherein the mass spectrometry analysis is carried out with HCCE matrix.

26. The method of claim 24, wherein enzymatic digestion is carried out with trypsin.

27. The cross-linking agent of claim 15, wherein R₁ is a phenoxy group.
